(19) United States
(12) Patent Application Publication
(10) Pub. No.: US 2006/0024367 A1
(43) Pub. Date: Feb. 2, 2006

(54) Controlled release alpha lipoic acid formulation with an inositol compound

(76) Inventor: Edward A. Byrd, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/199,919

(22) Filed: Aug. 8, 2005

Related U.S. Application Data
(63) Continuation-in-part of application No. 10/412,559, filed on Apr. 11, 2003.
Application No. 10/412,559 is a continuation of application No. 09/755,890, filed on Jan. 5, 2001, now Pat. No. 6,572,888.
Application No. 09/755,890 is a continuation-in-part of application No. 09/288,245, filed on Apr. 8, 1999, now Pat. No. 6,197,340.
Application No. 09/755,890 is a continuation-in-part of application No. 09/112,623, filed on Jul. 9, 1998, now abandoned.
(60) Provisional application No. 60/102,605, filed on Oct. 1, 1998.
(60) Provisional application No. 60/087,203, filed on May 28, 1998.

(51) Int. Cl. A61K 9/22; A61K 31/704; A61K 31/663; A61K 31/385

(52) U.S. Cl. 424/468; 514/25; 514/102; 514/440

(57) ABSTRACT

A biphasic formulation of an inositol compound and lipoic acid for oral administration is disclosed. The lipoic acid and the inositol compound are combined with excipient materials in such a way that those materials provide for an immediate release of a first portion of the active ingredients from the formulation followed by a gradual release of any remaining active ingredients in a manner which makes it possible to (1) quickly obtain a therapeutic level of the active ingredients; and (2) substantially increase the period of time over which therapeutic levels of the active ingredients are maintained relative to a quick release formulation. These features make it possible to use the formulation to reduce serum glucose levels and maintain those reduced glucose levels over time to treat diabetic polyneuropathy and thereby obtaining a range of desired therapeutic results.

### CROSS-REFERENCE TO RELATED APPLICATIONS

This application is a continuation-in-part application of earlier filed U.S. patent application Ser. No. 10/412,559, filed Apr. 11, 2003 which is a continuation of Ser. No. 09/755,890, filed Jan. 5, 2001 (now issued U.S. Pat. No. 6,572,888 issued Jun. 3, 2003) which is a continuation-in-part of earlier filed patent application Ser. No. 09/288,245, filed Apr. 8, 1999 (now issued U.S. Pat. No. 6,197,340 issued Mar. 6, 2001), which is a continuation-in-part of earlier filed provisional patent application Ser. No. 60/102,605, filed Oct. 1, 1998 and patent application Ser. No. 09/112,623, filed Jul. 9, 1998, which is the converted patent application of provisional patent application Ser. No. 60/087,203, filed May 28, 1998 to which we claim priority under 35 U.S.C. § 120 and § 119(e) each of which is incorporated herein by reference in their entirety.

### FIELD OF THE INVENTION

The invention relates generally to the treatment of diabetes mellitus, insulin resistance, metabolic syndrome, and polycystic ovary syndrome (PCOS) with an oral formulation which may be a controlled release oral formulation of pharmaceutically active compounds. More particularly the invention relates to an oral formulation of an inositol compound, e.g., inositol, or an inositol derivative or analog, combined with lipoic acid.

### BACKGROUND OF THE INVENTION

myo-Inositol is one of nine isomers of hexahydroxycyclohexane, and constitutes most of the naturally-occurring inositol of mammalian tissues. D-chiro-Inositol, an epimer of myo-inositol, is present in small amounts in mammalian tissues. D-chiro-Inositol is found in inositol phosphoglycans thought to be mediators of insulin signaling, as well as in certain mammalian glycosylphosphatidylinositol protein anchors. Diabetic patients excrete large amounts of D-chiro-inositol in urine.

Lipid soluble forms of thiamine include benfotiamine and prosultiamine. When these compounds are orally administered they provide greater bioavailability as compared to water soluble versions of conventional thiamine (see Greg et al., Internation. J. Clinical Pharm. And Therapeutics, Vol. 36, No. 4, pages 216-221 (1998)) Benfotiamine in combination with vitamine B has been used in the treatment of diabetic polyneuropathy. (See Stracke et al., Exp. Clin. Endocrinal Diabetes, vol. 104, pages 311-316 (1996)).

A compound known as α-lipoic acid was first isolated by Reed and coworkers as an acetate replacing factor. It is slightly soluble in water, and soluble in organic solvents. α-lipoic acid is a chiral molecule and is known by a variety of names, including thioctic acid; 1,2-diethylene-3 pentanoic acid; 1,2-diethylene-3 valeric acid; and 6,8-thioctic acid. α-lipoic acid was tentatively classified as a vitamin after its isolation, but it was later found to be synthesized by animals and humans. The complete enzyme pathway that is responsible for the de novo synthesis has not yet been definitively elucidated. Several studies indicate that octanoate serves as the immediate precursor for the 8-carbon fatty acid chain, and cysteine appears to be the source of sulfur. As a lipoamide, it functions as a cofactor in the multienzyme complexes that catalyze the oxidative decarboxylation of α-keto acids such as pyruvate, α-keto glutarate, and branched chain α-keto acids.

More recently, a great deal of attention has been given to possible antioxidant functions for α-lipoic acid, and its reduced form, dihydrolipoic acid (DHLA). Lipoate, or its reduced form, DHLA, reacts with reactive oxygen species such as superoxide radicals, hydroxyl radicals, hypochlorous acid, peroxyl radicals, and singlet oxygen. It also protects membranes by interacting with vitamin C and glutathione, which may in turn recycle vitamin E. In addition to its antioxidant activities, DHLA may exert prooxidant actions to reduction of iron. α-lipoic acid administration has been shown to be beneficial in a number of oxidative stress models such as ischemia-reperfussion injury (IRI), diabetes (both α-lipoic acid and DHLA exhibit hydrophobic binding to proteins such as albumin, which can prevent glycation reactions), cataract formation, HIV activation, neurodegeneration, and radiation injury. Furthermore, lipoate can function as a redox regulator of proteins such as myoglobin, prolactin, thioredoxin, and NF-κB transcription factor.

Lipoate may also have other activities. For example, DHLA has been found in vitro to be an anti-inflammatory agent which at the same time interferes with nitric oxide release from inflammatory macrophages and protects target cells from oxygen radical attack. V. Burkhart, Dihydrolipoic Acid Protects Pancreatic Islet Cells from Inflammatory Attack, Agents Actions 38:60 (1993). This document, and all other documents cited to herein, is incorporated by reference as if reproduced fully herein.

Lipoic acid is a coenzyme for several enzymes. Lipoic acid is a coenzyme for both α-keto acid dehydrogenase complex enzymes (i.e. pyruvate dehydrogenase complex and α-keto glutarate dehydrogenase complex), branched chain α-keto acid dehydrogenase complex, and the glycine cleavage system. In the enzyme system, the body forms a multi-enzyme complex involving lipoic acid, that breaks down molecules of pyruvate produced in earlier metabolism, to form slightly smaller, high energy molecules, called acetyl-coenzyme A. This results in molecules that can enter into a series of reactions called the citric acid cycle, or Krebs cycle, which finishes the conversion of food into energy. Essentially, lipoic acid stimulates basal glucose transport and has a positive effect on insulin stimulated glucose uptake.

Non-insulin dependent diabetes (NIDDM, or type 2 diabetes) is a worldwide health problem. According to the World Health Organization, an estimated 30 million people worldwide had diabetes in 1985. This number increased to 135 million people by 1995 and the WHO predicts a rise to 300 million people by 2025. The insidious nature of type 2 diabetes progression and medical complications that arise from hyperglycemia exact a heavy toll on the individual, healthcare resources, and society. As such, there is a continuing need for new formulations for use in treating diabetes, and preventing or delaying diabetic complications. The present invention addresses this need.

Literature

U.S. Pat. No. 6,492,339; Ostlund et al. (1993) Proc. Natl. Acad. Sci. USA 90:9988-9992; U.S. Pat. No. 6,492,341; U.S. Pat. No. 6,518,318.

### SUMMARY OF THE INVENTION

An oral formulation of an inositol compound and lipoic acid is disclosed, which formulation is comprised of these pharmaceutically active components alone or with one or more excipient materials. A wide range of different formulations of the two main active ingredients in quick release as well as biphasic and controlled release formulations will be apparent to those skilled in the art upon reading this disclosure. The formulation of lipoic acid and an inositol compound with an excipient material is designed to obtain a desired result, e.g. maintain sufficient blood levels of the inositol compound to support nerve regeneration and maintain sufficient blood levels of lipoic acid to reduce serum glucose levels. Both effects may combine to reduce the amount of medication (such as insulin and/or metformin hydrochloride) required to control symptoms of diabetes mellitus.

Formulations of the invention comprise two or more active components. The first is an inositol compound, e.g., inositol, or an inositol derivative, an inositol metabolite, an inositol analog, or an inositol-containing compound. Inositol includes chiro-inositol, e.g., D-chiro-inositol (also referred to as 1D-chiro-inositol or D-(+)-chiro-inositol). Examples of inositol derivatives and analogs include, but are not limited to, pinitol, e.g., D-pinitol. One, two or more different inositol compounds may be present together in the formulation or may be administered in separate oral formulations in the same treatment protocol of the same patient.

The second active component is lipoic acid which may be present as a racemic mixture, as the R-(+) enantiomer in amounts from 50% to 100% (of the lipoid acid component) or as the S-(−) enantiomer in amounts from 50% to 100% (of the lipoic acid component). If it is understood that if one enantiomer is present in an amount of more than 50% the other component is present in corresponding smaller percentage amounts. For example if the R-(+) enantiomer is present in amounts of 60%, 70%, 80%, 90% or 95% the S-(−) enantiomer is present in amounts of 40%, 30%, 20%, 10% or 5% respectively.

The formulation of the invention can be used not only to control blood glucose levels and treat diabetic polyneuropathy but for other complications of diabetics including diabetic neuropathy, diabetic nephropathy, and macrovascular disease. The formulation of the invention can also be used to treat insulin resistance, metabolic syndrome, and polycystic ovary syndrome (PCOS) The formulation of the invention makes it possible to obtain long term high plasma and tissue levels of an inositol compound. The formulation of lipoic acid and an inositol compound provide a unique complimentary and synergistic combination of active ingredients for treating a wide variety of manifestation of diabetes arising from the toxicity of chronically elevated plasma glucose.

One aspect of the invention is a biphasic formulation which provides a quick release of a portion of the active components of the formulation followed by controlled release of the remainder which increases the period of time that a therapeutic level of the inositol compound and lipoic acid are continuously maintained in the patient. The therapeutic level as well as the period of time over which that level must be maintained can vary between patient based on a range of factors such as the condition of the patient and the patient's reactivity to lipoic acid and the inositol compound. However, an oral formulation of the invention will maintain a therapeutic level over a period of time which is greater than that obtained with a conventional quick release formulation.

The ratio of active components to excipient material and the particular excipients used result in a formulation which allows the active components to be released quickly at first and thereafter in a controlled manner for absorption into the circulatory system. By maintaining a desired serum level of active components in blood serum the oral formulation of the invention achieves physiological effects which are superior to those obtained when higher serum levels are obtained for a short term with a quick release oral dosage formulation or a single dose injectable formulation.

By providing a biphasic formulation of active components the physiological effects are provided quickly at first to raise blood levels and then continually provided over a period of time resulting in improved nerve regeneration, reduced glucose levels and hemoglobinA_{1c} levels and thereby obtaining a range of associated health benefits. The controlled release formulation of the invention shows that highly desirable therapeutic effects can be obtained by maintaining a therapeutic blood serum level of the active components over a period of time which is meaningfully longer than that obtained with a quick release formulation and results are improved by maintaining such day after day over a period of 3 days, 7 days, 10 days, 30 days, 60 or more days.

A formulation of the invention will preferably obtain initial levels of lipoic acid at substantially the same rate as a quick release formulation and thereafter maintain therapeutic levels of lipoic acid over a period which is 10% or more, more preferably 50% or more and still more preferably 100% or more longer than a quick release formulation maintains therapeutic levels. To obtain a particularly preferred result the oral formulation of the invention will quickly release a sufficient amount of lipoic acid so as to quickly obtain a therapeutic level and thereafter release lipoic acid at a rate which substantially matches the rate at which the lipoic acid is being metabolized. Accordingly, a particularly preferred biphasic formulation is designed to (1) raise lipoic acid levels quickly to a therapeutic level; and (2) thereafter maintain a therapeutic level over a maximum amount of time based on the amount of lipoic acid in the formulation and to not significantly exceed the therapeutic level.

An aspect of the invention is an oral formulation of lipoic acid, and an inositol compound with excipient compounds which provide for controlled release.

Another aspect of the invention is a biphasic oral formulation of lipoic acid and an inositol which provides an immediate release of a first portion of the formulation to quickly raise blood serum levels to a therapeutic level and a controlled release of a second portion to maintain a therapeutic level over a maximum amount of time.

An advantage of the method and formulation of the invention is that by maintaining relatively low serum levels of lipoic acid and an inositol over long periods of time serum glucose levels are suppressed over long periods thereby inhibiting adverse effects which result from abnormally high serum glucose levels.

Another advantage of the invention is that by administering the formulation over long periods the patient is provided with a reduced risk of developing insulin resistance and/or diabetes mellitus.

Another aspect of the invention is that the formulation provides a method of treating type 2 diabetes, i.e. non-insulin-dependent diabetes mellitus (NIDDM).

Yet another aspect of the invention is that the lipoic acid may be present as a racemic mixture or with the R-(+) enantiomer present in amounts greater than 50% and constituting up to 100% of lipoic acid in the formulation.

An advantage of the invention is that a convenient oral delivery dosage form is used to obtain the results which are superior to a single dose injectable.

Another advantage of the invention is that glucose levels can be reduced and be maintained at levels substantially below levels without treatment via the present invention.

A feature of the invention is that the oral formulation may be a tablet, capsule, caplet, etc. containing any desired amount of lipoic acid.

Another aspect of the invention is that it may be formulated with one or more additional antidiabetic agents e.g. sulfonylureas; biguanides, α-glucosidase inhibitors, dipeptidyl peptidase (DPP) IV inhibitors, and thiazolidinediones, which agents may be formulated for quick release, controlled release or in a biphasic formulation.

Another aspect of the invention is a method of treatment whereby sustained low levels of lipoic acid blood serum over long periods continually stimulate basal glucose transport.

These and other objects, aspects, advantages, and features of the invention will become apparent to those persons skilled in the art upon reading the details of the invention as more fully described below.

### BRIEF DESCRIPTION OF THE DRAWING

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a conceptualized graph comparing a quick release oral dosage formulation to a biphasic oral dosage formulation of lipoic acid and an inositol wherein the amount released over time is graphed.

### DETAILED DESCRIPTION OF THE INVENTION

Before the present, formulations, methods and components used therein are disclosed and described, it is to be understood that this invention is not limited to particular compounds, excipients or formulations as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided are subject to change if it is found that the actual date of publication is different from that provided here.

### Definitions

The term “lipoic acid” is intended to mean α-lipoic acid which is a chiral molecule also known as thioctic acid; 1,2-diethylene-3 pentanoic acid; 1,2-diethylene-3 valeric acid; and 6,8-thioctic acid. Unless specified the term covers the racemic mixture as well as any other (non-50/50) mixture of the enantiomers including substantially pure forms of either the R-(+) or the S-(−) enantiomer. Further, unless specified otherwise the term covers pharmaceutically acceptable salts (e.g. Na and K salts) and amides, esters and metabolites of the acid. The molecule formula is C₈H₁₄O₂S₂ the molecular weight is 206.32 and it has a pKa of 4.7. In referring to pharmaceutically acceptable salts the term is intended to encompass a conventional term of pharmaceutically acceptable acid addition salts which refer to salts which retain the biological effectiveness and properties of the free-base form of the acid and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malconic acid, succinic acid, maleic acid, fumaric, tartaric acid, citric acid, benzoic acid, cinnamic acid, mandelic acid, methanesulfonic acid, ethanesulfonic acid, p-toluenesulfonic acid, salicylic acid and the like. The same is true with respect to amides, esters and metabolites that is those forms which can be formed and maintain biological effectiveness and not have significant undesirable biological properties.

As used herein, the terms “an inositol compound” and “an inositol” refer to D-(+)-chiro-inositol, and metabolites, analogs, and derivatives thereof, as well as inositol-containing compounds comprising D-chiro-inositol as part of a larger structure, which metabolites, analogs, derivatives, and inositol-containing compounds function to reduce blood glucose levels.

The term “excipient material” is intended to mean any compound forming a part of the formulation which is intended to act merely as a carrier i.e. not intended to have biological activity itself.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle.

The term “chemical degradation” is intended to mean that the lipoic acid active ingredient is subjected to a chemical reaction which disrupts its biological activity.

The terms “treating,” and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting it's development; or (c) relieving the disease, i.e. causing regression of the disease and/or it's symptoms or conditions. The invention is directed towards treating patient's suffering from a disease related to diabetes mellitus including adverse effects due to abnormally high levels of glucose as well as diabetic polyneuropathy and the effects of free radicals and/or oxidizing agents over long periods of time. The present invention is involved in preventing, inhibiting, or relieving adverse effects attributed to high levels of serum glucose over long periods of time and/or are such caused by free radicals or oxidizing agents present in a biological system over long periods of time.

The terms “individual,” “host,” “subject,” and “patient,” used interchangeably herein, refer to a mammal, including, but not limited to, murines, felines, simians, humans, mammalian farm animals, mammalian sport animals, and mammalian pets.

The terms “synergistic,” “synergistic effect,” and the like are used interchangeably herein to describe improved treatment effects obtained by combining controlled release lipoic acid formulations of the invention with an inositol compound and optionally with one or more other orally effective anti-diabetic compounds. Although a synergistic effect in some fields means an effect which is more than additive (e.g., one plus one equals three) in the field of treating diabetes and related diseases an additive (one plus one equals two) or less than additive (one plus one equals 1.3) effect may be synergistic. For example, if a patient has an abnormally high glucose level, e.g. 400 mg/dl, that patient's glucose level might be reduced to 300 mg/dl by the conventional orally effective antidiabetic compound. Further, at a different time the same patient with a glucose level of 400 mg/dl might be administered a different orally effective antidiabetic compound which compound reduced the patient's glucose levels from 400 to 300 mg/dl. However, if both orally effective antidiabetic compounds are administered to the patient one would not ordinarily expect an additive effect thereby obtaining a reduction to 200 mg/dl and may obtain no more of a reduction in glucose level than when either drug is administered by itself. If additive effects could always be obtained then diabetes could be readily treated in all instances by coadministering several different types of orally effective antidiabetic compounds until the disease is cured—but this approach is not an effective treatment. However, in connection with the present invention coadministration of formulations of controlled release lipoic acid with an inositol will obtain results which are synergistic, i.e. greater than the effects obtained by the administration of either composition by itself. The two active compounds may be further administered with one or more additional orally effective antidiabetic compounds such as metformin hydrochloride to obtain a further synergistic result.

The term “quick release formulation” refers to a conventional oral dosage formulation. Such a formulation may be a tablet, capsule, pill, liquid suspension or the like designed to provide for substantially immediate release of the active ingredient and includes enteric coated oral formulations which provide some initial protection to the active ingredient and thereafter allow substantially immediate release of substantially all the active ingredient. A quick release formulation is not formulated in a manner so as to obtain a gradual, slow, or controlled release of the active ingredient.

The terms “biphasic formulation,” “biphasic dosage form” and the like are used interchangeably here to describe any oral formulation with two different release rates. As an example, the biphasic formulation provides for an immediate release of a first portion of both the lipoic acid and the inositol compound followed by a slower, controlled and metered release of a second portion of the remainder of the lipoic acid and the inositol compound. Thus, a biphasic formulation of the invention preferably quickly raises blood levels to a therapeutic level of both active components and thereafter provides for a slower release which maintains the therapeutic level over a substantially longer time as compared to a quick release (10%, 50%, 100% or 200% longer) preferably without significantly exceeding the therapeutic level.

Thiamine or vitamin B₁ is C₁₂, H₁₇, ON₄, S HCl or thiamine hydrochloride. The compound is soluble in water and insoluble in ether and lipids. The RDA for vitamin B1 is about 1.2 mg per day, or 1.4 mg during pregnancy or lactation. Infants need more per body weight though less in total, about 0.5 mg per day. Thiamine needs are based on many factors; given good health, we need about 0.5 mg per 1,000 calories consumed, since B1 is required for energy metabolism. So our needs are based on body weight, calorie consumption, and the amount of vitamin B1 synthesized by intestinal bacteria, which can vary greatly from person to person.

Thiamine is a coenzyme for the decarboxylation of pyruvate and the oxidation of alpha keto-glutamic acid. Lipoic acid which is formed in the liver is also required for the reactions. Patients with liver disease may show signs of B1 deficiency, possibly because of deficient synthesis of lipoic acid. In vitro, thiamine deficiency produces accumulation of pyruvate and lactate, reduction of acetate, citrate and alpha-keto-glutarate and reduced acetylcholine synthesis. Any of these metabolic changes could be involved in dysfunction.

The term “lipid soluble thiamine” is used here to cover derivatives of thiamine with higher solubility in lipids as compared to thiamine, e.g. 10%, 50%, 100%, 200% or 10 times or more, more soluble in lipids as compared to thiamine. Specific lipid soluble thiamines include benfotiamine and prosultiamine. The term as used here is intended to cover pharmaceutically acceptable salts, acids, and esters thereof.

### Formulation in General

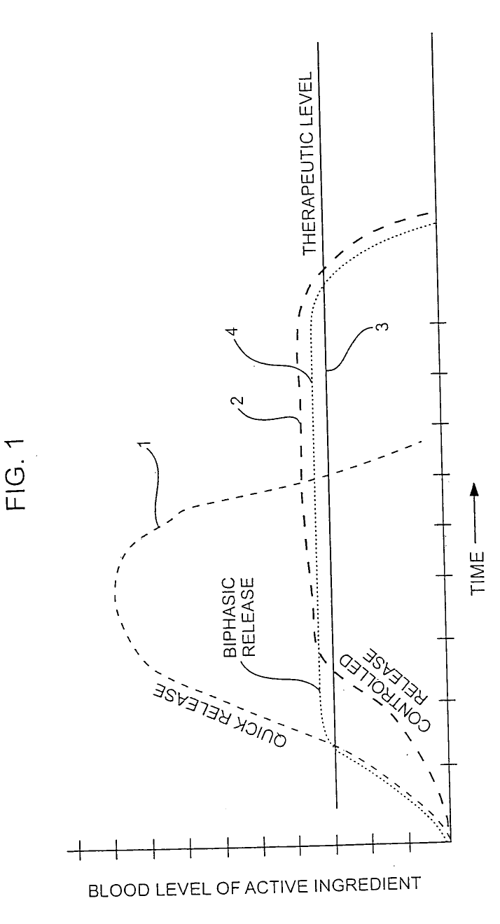

Referring to FIG. 1 which is a conceptualized graph provided to show a comparison between a theoretical quick release and theoretical biphasic oral formulation. The graph shows the amount of the active components in the patient over time. The light dashed line 1 is of a theoretical quick release oral formulation showing that the level of active component rises and falls quickly. The bold dashed line 2 is of a theoretical controlled release formulation which initially rises more slowly as compared to the quick release formulation after reaching the therapeutic level shown by the solid line 3 it enters the controlled release phase and maintains a level at or just above the therapeutic level until no more active component is available in the dosage form. At this point the line drops to zero quickly as there is no more active component in the formulation for release and remaining active component is metabolized.

The dotted line 4 shows the release rate of a biphasic formulation. In the first phase, release rate of the active component is substantially the same as the quick release formulation. The biphasic formulation reaches the therapeutic level at substantially the same time as the quick release formulation does. Thereafter, the biphasic formulation begins a slower release as compared to the quick release formulation. For example, the rate of release of active component in the second phase is substantially equal to the rate at which the active components are metabolized. As with the controlled release formulation the object is to keep the level as close to the therapeutic level as possible for as long as possible.

Formulations

The present invention provides controlled release formulations comprising an inositol compound. The present invention further provides formulations comprising a lipoic acid and an inositol compound. The formulations are useful for regulating blood glucose levels in an individual; and for treating disorders relating to or resulting from abnormal blood glucose levels.

Inositol Compound Formulations

The present invention provides controlled release formulations comprising an inositol compound (“an inositol compound controlled release formulation”). In some embodiments, a subject inositol compound controlled release formulation is a quick release formulation. In some embodiments, a subject inositol compound controlled release formulation is a biphasic release formulation. In some embodiments, a subject inositol compound controlled release formulation is a slow release formulation. Suitable quick release, biphasic release, and slow release formulations are described in detail below.

The inositol compound is generally present in the formulation such that a unit dosage form contains the inositol compound in an amount of from about 50 mg to about 5000 mg, e.g., from about 50 mg to about 100 mg, from about 100 mg to about 250 mg, from about 250 mg to about 500 mg, from about 500 mg to about 750 mg, from about 750 mg to about 1000 mg, from about 1000 mg to about 1500 mg, from about 1500 mg to about 2000 mg, from about 2000 mg to about 2500 mg, from about 2500 mg to about 3000 mg, from about 3000 mg to about 3500 mg, from about 3500 mg to about 4000 mg, from about 4000 mg to about 4500 mg, or from about 4500 mg to about 5000 mg.

Suitable inositol compounds include, but are not limited to, D-chiro-inositol; D-chiro-inositol phosphates; D-chiro-inositol esters, e.g., D-chiro-inositol acetates; D-chiro-inositol ethers, e.g., D-chiro-inositol lower alkyl ethers; D-chiro-inositol acetals; and D-chiro-iniositol ketals. Suitable inositol compounds include compounds that contain a D-chiro-inositol moiety as part of a larger structural composition. Suitable D-chiro-inositol containing compounds include, but are not limited to, the following: polysaccharides containing D-chiro-inositol and one or more additional sugars, such as glucose, galactose and mannose, or derivatives thereof, such as glucosamine, galactosamine and mannitol; D-chiro-inositol phospholipids; and complexes or chelates of D-chiro-inositol with one or more metal ions and the like. A non-limiting example of such a D-chiro-inositol compound is 2-O-α-D-galactopyranosyl-D-chiro-inositol. Suitable inositol compounds include D-chiro-inositol, a D-chiro-inositol-phosphate, pinitol, ciceritol, 1D-2-O-α-D-galactopyranose, and a fagopyritol. Fagopyritols include Fagopyritol A1, Fagopyritol A3, Fagopyritol A3, Fagopyritol B1, Fagopyritol B2, Fagopyritol B3. See, e.g., U.S. Pat. No. 6,492,341 for a description of various fagopyritols and methods of preparing same. Suitable pinitol compounds include pinitol; and pinitol derivatives and metabolites, e.g., pinitol glycosides, pinitol phospholipids, esterified pinitol, lipid-bound pinitol, pinitol phosphates, pinitol phytates, and galactopinitol. Methods of making D-chiro inositol are known in the art; see, e.g., U.S. Pat. Nos. 5,600,014; and 5,932,774. The structure of ciceritol a pinitol digalactoside, is described in, e.g., Bernabe et al. (1993) J. Agric. Food Chem., 41 870-872. The structures of additional inositol compounds are shown in Kornienko, et al. ((1998) Carbohydrate Res. 310:141-144).

Lipoic Acid/Inositol Compound Formulations

The present invention provides formulations comprising a lipoic acid and an inositol compound. In one aspect of the invention, the two active components are separately formulated with excipient and thereafter combined. This is done because lipoic acid is metabolized more quickly as compared to inositols in general. In one embodiment the inositol compound is all in a quick release formulation and combined with lipoic acid in a biphasic formulation, i.e. both quick release and controlled release formulation. Such a formulation obtains enhanced bioavailability of the inositol compound and increases the length of time that therapeutic levels of lipoic acid are maintained via the biphasic release formulation of that component.

Suitable inositol compounds are as described above. Suitable inositol compounds include, but are not limited to, D-chiro-inositol; D-chiro-inositol phosphates; D-chiro-inositol esters, e.g., D-chiro-inositol acetates; D-chiro-inositol ethers, e.g., D-chiro-inositol lower alkyl ethers; D-chiro-inositol acetals; and D-chiro-iniositol ketals. Suitable inositol compounds include compounds that contain a D-chiro-inositol moiety as part of a larger structural composition. Suitable D-chiro-inositol containing compounds include, but are not limited to, the following: polysaccharides containing D-chiro-inositol and one or more additional sugars, such as glucose, galactose and mannose, or derivatives thereof, such as glucosamine, galactosamine and mannitol; D-chiro-inositol phospholipids; and complexes or chelates of D-chiro-inositol with one or more metal ions and the like. Suitable inositol compounds include D-chiro-inositol, a D-chiro-inositol-phosphate, pinitol, ciceritol, 1D-2-O-α-D-galactopyranose, and a fagopyritol. Fagopyritols include Fagopyritol A1, Fagopyritol A3, Fagopyritol A3, Fagopyritol B1, Fagopyritol B2, Fagopyritol B3. See, e.g., U.S. Pat. No. 6,492,341 for a description of various fagopyritols and methods of preparing same. Suitable pinitol compounds include pinitol; and pinitol derivatives and metabolites, e.g., pinitol glycosides, pinitol phospholipids, esterified pinitol, lipid-bound pinitol, pinitol phosphates and pinitol phytates. Methods of making D-chiro inositol are known in the art; see, e.g., U.S. Pat. Nos. 5,600,014; and 5,932,774. The structure of ciceritol a pinitol digalactoside, is described in, e.g., Bernabe et al. (1993) J. Agric. Food Chem., 41 870-872.

The inositol compound is generally present in the formulation such that a unit dosage form contains the inositol compound in an amount of from about 50 mg to about 5000 mg, e.g., from about 50 mg to about 100 mg, from about 100 mg to about 250 mg, from about 250 mg to about 500 mg, from about 500 mg to about 750 mg, from about 750 mg to about 1000 mg, from about 1000 mg to about 1500 mg, from about 1500 mg to about 2000 mg, from about 2000 mg to about 2500 mg, from about 2500 mg to about 3000 mg, from about 3000 mg to about 3500 mg, from about 3500 mg to about 4000 mg, from about 4000 mg to about 4500 mg, or from about 4500 mg to about 5000 mg.

The formulation of the invention is in many embodiments an oral dosage formulation which may be in any suitable oral form including tablets, pills, capsules, caplets, lozenges, liquid suspensions, etc. The dosage may be of any desired size in terms of the two active ingredients. However, sizes for the combined two active ingredients in a range of about 50 mg to about 1,000 mg are generally used, or for example 100 mg to 500 mg or alternatively about 200 mg to about 400 mg.

Therapeutic results can, in some cases be obtained with the inositol compound present in a dosage form such as a capsule in an amount of about 50 mg to 1,000 mg. The dosage formulation of the invention can be taken once a day or 2, 3, 4, 5 or more times a day. These amounts can be total amounts per day or can be modified to be amounts per day per 1,000 calories consumed by the patient.

Although the ratio of lipoic acid to inositol compound can vary, the ratio may be about 10:1, 8:1, 6:1, 4:1, 2:1, 1:1, 1:2, 1:4, 1:6, 1:8, 1:10 of lipoic: inositol compound. A 1:1 ratio ±20% is acceptable.

The biphasic formulation is constructed to hold the active components in different combinations of excipients. Preferably the center portion of the formulation will be produced in accordance with the examples provided here. The outer portion of the formulation could be the active components alone or mixed with any excipients in the same proportional amounts generally used by those of ordinary skill in the art in producing a conventional quick release formulation.

The quick release portion may comprise from about 10% to about 50% of the active components in the formulation or preferably about 20% to about 30% and more preferably about 25% of the active components in the formulation.

The amount a patient will need to obtain an optimum therapeutic effect will vary with a number of factors known to those skilled in the art e.g. the size, age, weight, sex and condition of the patient. The patient may begin with daily doses of about 300 mg of lipoic acid and 300 mg of an inositol compound and determine if desired results are obtained, e.g. glucose levels are reduced to acceptable levels. If the desired results are not obtained in 7-10 days the daily dosage amount can be increased in increments for both of the active components. For example, both the inositol compound and lipoic acid can be increased in amounts of 100 to 300 mg/day up to any useful amount e.g. 2,000 mg/day of each of the active components. Longer time periods such as 3 month, 6 months, 12 months or longer may be required to observe improved results in other areas such as decreases in diabetic polyneuropathy.

A suggested dosage is to administer two tablets in the morning and administer one tablet four hours later and repeat daily over five or more days where the tablet comprise 300 mg of lipoic acid and 300 mg of an inositol compound. The larger initial dosage has been found effective in obtaining a desired effect which after being obtained can be maintained by a lower dose. Thus, a biological system may be “kick started” by a high therapeutic level and then maintained at a lower level which is also therapeutic in terms of obtaining a desired result. In a particularly preferred formulation the inositol compound is present as 200 mg of quick release and 100 mg of controlled release and 100 mg of the 300 mg of lipoic acid is in a quick release formulation in the outer shell of the tablet and the inner 200 mg is in a controlled release formulation.

The manufactured compound α-lipoic generally exists as a 50/50 or racemic mixture of R-(+)-α-lipoic acid and S-(−)-α-lipoic acid. The R-(+) enantiomer is the naturally produced biological form of the compound and as such is believed to be largely responsible for obtaining the physiological effect of the lipoic acid component. Thus, the lipoic acid ingredient of the formulation of the present invention may be 100% R-(+) enantiomer. However, the active ingredient may be present in any mix of the two enantiomers e.g. 10% S-(−) and 90% R-(+); 25% S-(−) and 75% R-(+). Further, it should be noted that even though the R-(+) enantiomer is believed to be the more active the S-(−) enantiomer may possess unique properties which make inclusion of the S-(−) enantiomer important in any formulation used in treatment. Unless stated otherwise information disclosed here refers to formulations containing a racemic mixture. If the active ingredient is not a racemic mixture then some adjustment may be needed in the formulation in order to account for the greater activity of the R-(+) enantiomer as well as the slightly longer half life of the R-(+) enantiomer compared to the S-(−) enantiomer.

A typical formulation contains about 50-70% by weight active ingredient with the remainder being excipient material. The quick release portion of the formulation may comprise 100% active components or a very small amount e.g. 5-10% by weight of excipient. The controlled release portion of the formulation may comprise 55% to 65% active ingredient and more preferably about 60% active ingredient by weight. Thus, a particularly preferred oral formulation of the invention comprises about 300 mg of lipoic acid, 300 mg of an inositol compound and about 200 mg of excipient material. Human patients generally eat during the day and sleep at night. Eating causes increased glucose levels. Accordingly, it is generally preferable to give a larger dose of lipoic acid at the beginning of the day. This may include 1 to 6 tablets of 150 mg of lipoic acid and 150 mg inositol compound. Later in the day (about 4 hours) the patient will take an additional dose which is generally smaller or about one half of the morning dose.

The formulation is characterized by (a) protecting the active ingredient (to the extent required) from chemical degradation in a patient's gastrointestinal tract and (b) releasing the active ingredient in a controlled manner. By gradually releasing the active ingredient the serum levels of the active components obtained are (1) lower than those obtained with single dose injectable or a non-controlled release formulation; and (2) maintained over longer periods of time than obtained with single dose injectable or a non-controlled release formulation. A preferred biphasic formulation of the invention releases active ingredient so as to obtain a blood serum level in a human patient in a range of about 25 ng/ml to 2,500 ng/ml of plasma for both inositol compound and lipoic acid. The range is preferably about 50 ng/ml to 2,000 ng/ml of plasma and more preferably about 1,800 ng/ml of plasma −20% for both inositol compound and lipoic acid. The plasma level that is therapeutic will vary somewhat from patient to patient depending on factors such as the weight, sex and age and condition of the patient and will vary further depending on the therapy or treatment being sought.

Some characteristics of lipoic acid are (1) it is non-toxic at relatively high levels, i.e. levels well in excess of therapeutic levels; and (2) lipoic acid is quickly metabolized by human patients. The present invention relies in part on the discovery that lipoic acid provides desirable therapeutic results even at very low levels provided those low levels are maintained over an extended period of time whereas therapeutic results are not obtained (even with higher levels) if the therapeutic level is not maintained over a sufficiently long period of time. Further, the present invention relies in part on the discovery that therapeutic results are further improved if the delivery of lipoic acid is administered over a period of five or more, preferably thirty or more consecutive days with long periods (four hours, eight hours, or 12 hours or more) of therapeutic levels of lipoic acid being obtained on each of the days. Another aspect of the invention is the synergistic effect obtained by confirming the effects of lipoic acid with an inositol compound. Yet another aspect of the invention is the improved bioavailability of the inositol compound when used in a controlled release formulation.

One aspect of the invention is that a range of highly desirable therapeutic effects are obtained even when both the inositol compound and lipoic acid blood serum levels are maintained in a range well below those previous used. The present invention could obtain desired therapeutic effects with higher levels of both the inositol compound and lipoic acid in blood serum. However, at least minimum levels would need to be constantly maintained over a long period of time (4 hours or more per day) for a plurality of days to obtain the desired results. When the oral dosage form is designed to obtain the lowest possible therapeutic level over the longest possible time period the results obtained are maximized and the amount of drug needed is minimized.

The blood plasma level of both the inositol compound and lipoic acid obtained via the present invention is insufficient to obtain a desired therapeutic effect if that level is maintained for only a short period of time. The amount of time and the level needed can vary based on factors such as the condition of the patient and the results desired. In general, longer periods at a sustained level are preferred to short periods and large fluctuation in levels. By using the biphasic oral formulation of the invention therapeutic lipoic acid blood plasma levels can be maintained over 8 hours or more, preferably over 12 hours or more and more preferably over 16 hours or more per day. Further, those plasma levels of both the inositol compound and lipoic acid over these periods of time are repeatedly obtained on consecutive days, preferably weeks or months and more preferably continuously over any period during which the patient would benefit from reduced serum glucose levels—which may be the remainder of the patient's life.

To obtain the desired results a formulation of the invention needs to start with a sufficient amount of both the inositol compound and lipoic acid such that it is capable of releasing enough of both the inositol compound and lipoic acid per unit of time to obtain the desired serum levels of both the inositol compound and lipoic acid while compensating for both the inositol compound and lipoic acid which is metabolized. To obtain the desired results the biphasic formulation provides an initial release of both the inositol and lipoic acid quickly and thereafter provides a gradual release which slows over the useful life of the formulation. Desired results can be obtained with a single phase controlled release formulations where the release may be gradual from the beginning. In either case there is preferably a gradual slowing of the rate of release which is compensated for in that some of the previously released amounts of both the inositol and lipoic acid remain in the blood serum unmetabolized.

A preferred oral formulation is a tablet which is designed to provide an initial quick release of a portion of both the inositol compound and lipoic acid, e.g. about 25% and thereafter dissolve gradually over a period of about 8 hours. As the tablet dissolves its reduced size will release smaller and smaller amounts of both the inositol compound and lipoic acid per unit of time. However, because the individual's system already contains a therapeutic level of both the inositol compound and lipoic acid the slower release rate is sufficient to match the rate of both the inositol compound and lipoic acid being metabolized and such will result in maintaining a relatively constant therapeutic level as shown in FIG. 1. At the end of the time when release of both the inositol compound and lipoic acid is no longer taking place (e.g. about 4 to 8 hours) another tablet is administered and the process is repeated. To obtain the benefits of the invention the process is continually repeated over a plurality of days, weeks, months or years. By maintaining a minimal both the inositol compound and lipoic acid blood serum level of both the inositol compound and lipoic acid over time a patient's abnormally high serum glucose levels are reduced and the long term adverse effects of elevated serum glucose levels are avoided.

In some embodiments, a subject formulation comprising a lipoic acid and an inositol compound provides for total plasma lipoic acid (LAₜₒₜₐₗ) concentration of about 1800 ng/ml. In some embodiments, a subject formulation provides for a plasma LAₜₒₜₐₗ concentration of from about 1600 ng/ml to about 1800 ng/ml in a period of time of from about 10 minutes to about 40 minutes, e.g., from about 10 minutes to about 15 minutes, from about 15 minutes to about 20 minutes, from about 20 minutes to about 30 minutes, or from about 30 minutes to about 40 minutes, following administration of the formulation. In some embodiments, a subject formulation provides for a plasma LAₜₒₜₐₗ concentration of from about 1600 ng/ml to about 1800 ng/ml in a period of time of from about 10 minutes to about 40 minutes, e.g., from about 10 minutes to about 15 minutes, from about 15 minutes to about 20 minutes, from about 20 minutes to about 30 minutes, or from about 30 minutes to about 40 minutes, following administration of the formulation; followed by maintenance of the plasma LAₜₒₜₐₗ concentration at a level of from about from about 1600 ng/ml to about 1800 ng/ml over a period of time ranging from about one hour to about 48 hours, e.g., from about one hour to about two hours, from about two hours to about four hours, from about four hours to about six hours, from about six hours to about eight hours, from about eight hours to about ten hours, from about ten hours to about 12 hours, from about 12 hours to about 16 hours, from about 16 hours to about 24 hours, from about 24 hours to about 36 hours, or from about 36 hours to about 48 hours.

### Three Ingredient Formulations

The present invention provides formulations comprising lipoic acid, an inositol compound, and a third therapeutic agent. Suitable third therapeutic agents include, but are not limited to, an anti-diabetic agent (e.g. acarbose, sulfonylureas, biguanides, PPARγ agonists, PPARα/γ dual agonists, thiazolidinediones, DPP IV inhibitors), thiamine, and benfotiamine.

Lipoic Acid, Inositol Compound, Anti-Diabetic Agent

Lipoic acid acts directly on muscle cells to stimulate glucose transport. The effect of both the inositol compound and lipoic acid on serum glucose reduction obtained with lipoic acid may be sufficient for some patients. However, if an insufficient glucose lowering effect results the amount of both the inositol compound and lipoic acid may be supplemented with one or more orally effective antidiabetic agents selected from the group consisting of sulfonylureas, biguanides, thiazolidinediones, DPP-IV inhibitors, PPARγ agonists, PPAR α/γ dual agonists, and α-glucosidase inhibitors.

Thus, in some embodiments, the instant invention provides formulations comprising a lipoic acid, an inositol compound, and at least one additional anti-diabetic agent. Suitable anti-diabetic agents include, but are not limited to, thiazolidinediones, e.g., Avandia® (rosiglitazone maleate), agents of the sulfonylurea class; biguanides, e.g. metformin (Glucophage®); alpha-glucosidase inhibitors, e.g., acarbose (Precose®), miglitol, etc.; peroxisome proliferators activated receptor (PPAR) agonists; dipeptidyl peptidase IV (DPP-IV) inhibitors; and the like.

Suitable sulfonylureas include tolbutamide and glipizide and related compounds such as Amaryl, Pandin and Starlix. These drugs target pancreatic beta cells and stimulate these cells to release insulin. Suitable biguanides include compounds such as metformin, phenformin and buformin. These compounds act on the liver to decrease hepatic glucose output and on the intestine to block glucose uptake into the blood. Suitable thiazolidinediones include compounds such as pioglitazone, englitazone, MCC-555, rosiglitazone, and the like. These compounds are believed to sensitize muscle and fat cells to insulin. A thiazolidinedione may be together in the same dosage formulation as the lipoic acid/inositol compound formulation, or in a separate formulation administered at the same or different time.

Suitable DPP-IV inhibitors include, but are not limited to, an α-aminoacyl pyrrolidide, an α-aminoacyl thiazolidide, an α-aminoacyl pyrrolidinenitrile, an α-aminoacyl thiazolidinenitrile, tetrahydroisoquinolin-3-carboxamide derivatives, N-substituted 2-cyanopyroles and -pyrrolidines, N-(N′-substituted glycyl)-2-cyanopyrrolidines, N-(substituted glycyl)-thiazolidines, N-(substituted glycyl)-4-cyanothiazolidines, amino-acyl-borono-prolyl-inhibitors, cyclopropyl-fused pyrrolidines and heterocyclic compounds. Suitable DPP-IV inhibitors include those discussed in, e.g, WO 97/40832; WO 98/19998; U.S. Pat. No. 5,939,560; Bioorg. Med. Chem. Lett., 6(10), 1163-1166 (1996); Bioorg. Med. Chem. Lett., 6(22), 2745-2748 (1996); U.S. Patent Publication No. 2005/0107390; U.S. Pat. No. 6,380,398, U.S. Pat. No. 6,011,155; U.S. Pat. No. 6,107,317; U.S. Pat. No. 6,110,949; U.S. Pat. No. 6,124,305; U.S. Pat. No. 6,172,081; WO 95/15309, WO 99/61431, WO 99/67278, WO 99/67279, DE 198 34 591, WO 97/40832, DE 196 16 486 C₂, WO 98/19998, WO 00/07617, WO 99/38501, WO 99/46272, WO 99/38501, WO 01/68603, WO 01/40180, WO 01/81337, WO 01/81304, WO 01/55105, WO 02/02560 and WO 02/14271. In some embodiments, a DPP-IV inhibitor is a compound of Formula I as set forth in U.S. Patent Publication No. 2005/0107390. In some embodiments, a DPP-IV inhibitor is a compound of Formula I or Formula II as set forth in U.S. Patent Publication No. 20040209891. A DPP-IV inhibitor compound may be together in the same dosage formulation as the lipoic acid/inositol compound formulation, or in a separate formulation administered at the same or different time.

Suitable PPAR-αagonists include fenofibric acid derivatives (gemfibrozil, clofibrate, fenofibrate and bezafibrate). Suitable PPAR-y agonists include the glitazones (e.g. pioglitazone, englitazone, MCC-555, rosiglitazone, and the like); Farglitazar; Suitable PPAR α/γ dual agonists include, but are not limited to, KRP-297 (Fajas, 1997, J. Biol. Chem., 272:18779-18789); DRF-2725 and AZ-242 (Lohray, et al., 2001, J. Med. Chem., 44:2675-2678; Cronet, et al., 2001, Structure (Camb.) 9:699-706). KRP-297 has the following structure:

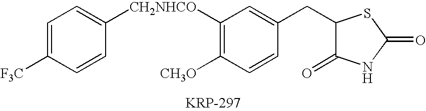

A PPAR agonist may be together in the same dosage formulation as the lipoic acid/inositol compound formulation, or in a separate formulation administered at the same or different time.

Although all or any orally effective antidiabetics can be formulated with or administered along with a formulation of the invention, in some embodiments, it is preferable to administer metformin (particularly metformin Hydrochloride tablets sold as Glucophage7) with controlled release formulations of the invention comprising therapeutically effective amounts of both lipoic acid and an inositol compound. Some particularly preferred formulations include 300 mg lipoic acid (racemic or R(+) α lipoic acid), 300 mg an inositol compound such as D-chiro-inositol or pinitol and 500 mg of metformin hydrochloride or, if a larger dose is needed, 600 mg of lipoic acid, 600 mg of an inositol and 1,000 mg of metformin hydrochloride.

In some embodiments, the anti-diabetic agent is miglitol (3,4,5-piperidinetriol, 1-(2-hydroxyethyl)-2-(hydroxymethyl)-, [2R-(2α, 3β, 4α, 5β)]) or Glyset® (miglitol; N-hydroxyethyl-DNJ). Miglitol (N-hydroxyethyl-DNJ) is described in U.S. Pat. No. 4,639,436. Miglitol has the structure shown in Formula I:

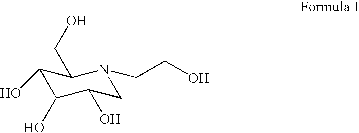

A suitable dosage of an α-glucosidase inhibitor contains an amount of from about 10 mg to about 100 mg miglitol, e.g., 10 mg, 15 mg, 20 mg, 25 mg, 30 mg, 40 mg, 50 mg, 60 mg, 70 mg, 75 mg, 80 mg, 90 mg, or 100 mg miglitol. Typically, miglitol is administered orally tid. Miglitol may be together in the same dosage formulation as the lipoic acid/inositol compound formulation, or in a separate formulation administered at the same or different time.

In some embodiments, the agent is acarbose (O-4,6-dideoxy-4-[[(1S,4R,5S,6S)-4,5,6-trihydroxy-3-(hydroxymethyl)-2-cyclohexen-1-yl]amino]-α-D-glucopyranosyl-(1→4)-O-α-D-glucopyranosyl-(1→4)-D-glucose), or Precose®. Acarbose is described in U.S. Pat. No. 4,904,769. In some embodiments, acarbose is a highly purified form of acarbose (see, e.g., U.S. Pat. No. 4,904,769). Acarbose has the structure shown in Formula II:

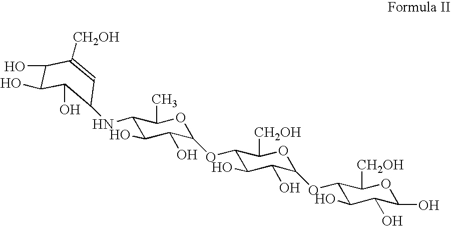

A suitable dosage of an α-glucosidase inhibitor contains an amount of from about 10 mg to about 100 mg acarbose, e.g., 10 mg, 15 mg, 20 mg, 25 mg, 30 mg, 40 mg, 50 mg, 60 mg, 70 mg, 75 mg, 80 mg, 90 mg, or 106 mg acarbose. Typically, acarbose is administered orally tid. Acarbose may be together in the same dosage formulation as the lipoic acid/inositol compound formulation, or in a separate formulation administered at the same or different time.

Additional enhanced effects may be obtained by taking a formulation of the invention along with vitamin C and/or vitamin E. For example a patient might take 900 mg/day of lipoic acid 900 mg/day of an inositol, 1,000 to 3,000 mg/day of vitamin C and 400 to 800 mg/day of vitamin E.

Example 10 provides specific examples of patients who underwent coadministration of controlled release lipoic acid formulations of the present invention in combination with other treatments conventionally used to lower serum glucose levels. The synergistic effects were obtained, i.e. the combination of lipoic acid controlled release formulations of the invention with other therapeutic agents obtained results which were greater than results which might be expected with the administration of either composition by itself.

The inositol compound and optional antidiabetic component may be (1) solely in the quick release portion of the formulation; (2) solely in the controlled release portion of the formulation; or (3) in both portions of the biphasic formulation with any amount in either phase of the formulation.

Lipoic Acid, Inositol Compound, Thiamine Formulations

In other embodiments, the instant invention provides formulations comprising a lipoic acid, an inositol compound, and thiamine. In some embodiments, the thiamine is a lipid-soluble thiamine. In some embodiments, the lipid-soluble thiamine is benfotiamine.

The inositol compound and optional thiamine component may be (I) solely in the quick release portion of the formulation; (2) solely in the controlled release portion of the formulation; or (3) in both portions of the biphasic formulation with any amount in either phase of the formulation.

### Excipient Material

Examples provided here show that formulations of the invention may comprise different amounts and ratios of active ingredient and excipient material. Further, different excipients can be used. Particularly preferred excipients and amounts used are recited in the Examples. However, upon reading the disclosure those skilled in the art will come to understand the general concepts of the invention and will recognize that other excipients, amounts, ratios and combinations might be used to obtain the results first shown here.

The type and amount of excipient material is added to obtain a formulation with two important characteristics. First, the resulting formulation protects the active ingredient from chemical degradation in the patient's gastrointestinal tract. Although the formulation need not protect 100% of the lipoic acid and/or inositol compound from degradation to come within the scope of the invention it may protect 90% or more, preferably 95% or more and more preferably 99% or more of the lipoic acid and/or inositol compound from degradation. Although multiple doses of an oral formulation could be taken it is preferable to design the dosage such that a single dose is taken at each dosing event—preferably three times a day and more preferably twice a day. The better the active ingredient is protected from degradation the less active ingredient is needed in the original dosage thereby reducing manufacturing costs and increasing profits. The formulation must protect at least as much of the dose as is needed to obtain a pharmacological effect and preferably obtain the desired treatment results, e.g. maintaining desired lipoic acid and inositol serum levels needed to obtain therapeutic results, e.g., a reduced serum glucose level over time.

Another desired characteristic of the formulation is that it does not release all of the active ingredients at one time but rather releases the active ingredients gradually over time at a controlled rate of release which rate is preferably constant over 4 hours or more. This is particularly important for the lipoic acid component because (1) lipoic acid has a relatively short half life and (2) a desired level of both inositol and lipoic acid in blood serum must be maintained over a long period to obtain the desired effect. If all of the lipoic acid is released at once it will all enter the circulatory system at once and be metabolized in the liver thereby causing the lipoic acid serum level to drop below the desired level. When this occurs the effect on reducing glucose levels is suboptimal.

These examples are more generally of the controlled release core phase of the biphasic tablets. The quick release outer phase can be manufactured using pure lipoic acid and inositol compound alone or with minimal excipients.

### Typical Formulations

A typical formulation of the invention will contain about 25% to 50% by weight of lipoic acid and 25% to 50% of inositol and a particularly preferred formulation will comprise 35% by weight of lipoic acid and 35% inositol and 30% carrier. Assuming a formulation with 35% by weight of lipoic acid 35% by weight of inositol with the remaining 30% being excipient material there are a number of possible components which could be used to make up that 30%. A generalized and specific description of such is provided below:

| | | |
|---|---|---|
| (1) | lipoic acid | 35% |
| | Inositol compound | 35% |
| | Organic polymer | 30% |
| | TOTAL | 100% |
| (2) | lipoic acid | 35% |
| | Inositol compound | 35% |
| | Organic polymer | 24.5% |
| | Inorganics | 5.5% |
| | TOTAL | 100% |
| (3) | lipoic acid | 35% |
| | Inositol compound | 35% |
| | Organic polymer | 20%-30% |
| | Inorganics | 10% |
| | | or less |
| | TOTAL | 100% |
| (4) | lipoic acid | 45% |
| | Inositol compound | 25% |
| | microcrystalline cellulose | 9% |
| | cellulose acetate phthalate aqueous | |
| | dispersion | 10% |
| | Polyvinylpyraolidone | 3% |
| | ethyl acetate | 2.5% |
| | hydrous magnesium silicate (talc) | 1% |
| | carboxy methyl ether | 4% |
| | magnesium stearate | 0.5% |
| | TOTAL | 100% |
| (5) | lipoic acid | 25% |
| | Inositol | 45% |
| | microcrystalline cellulose | 10-20% |
| | cellulose acetate phthalate aqueous | |
| | dispersion | 5-15% |
| | Polyvinylpyraolidone | 1-5% |
| | ethyl acetate | 1-5% |
| | Hydrous magnesium silicate (talc) | 0.5-3% |
| | Carboxy methyl ether | 1-5% |
| | magnesium stearate | 0.5-1.5% |
| | TOTAL | 100% |
| (6) | R-(+)-α-lipoic acid | 35% |
| | Pinitol | 25% |
| | microcrystalline cellulose, NF (Avicel PH 101) | 9% |
| | Aquacoat CPD-30 (30% solids w/w) | 10% |
| | Plasdone K29/32, USP | 3% |
| | Carbopol 974P, NF | 2.5% |
| | Talc, USP | 1.0% |
| | croscarmellose sodium, NF (Ac, di-Sol) | 4.0% |
| | Magnesium Stearate, NF | 0.5% |
| | TOTAL | 100% |
| (7) | R-(+)-α-lipoic acid | 40% |
| | Pinitol | 30% |
| | microcrystalline cellulose, NF (Avicel PH 101) | 10-20% |
| | Aquacoat CPD-30 (30% solids w/w) | 5-15% |
| | Plasdone K29/32, USP | 1-5% |
| | Carbopol 974P, NF | 1-5% |
| | Talc, USP | 0.5-3% |
| | croscarmellose sodium, NF (Ac, di-Sol) | 1-5% |
| | Magnesium Stearate, NF | 0.5-1.5% |
| | TOTAL | 100% |

Those skilled in the art will recognize that there are endless possibilities in terms of formulations and that a margin of error e.g. ±20% or more preferably ±10% should be accounted for with each component. Even if the formulations are limited to the relatively few compounds shown above the formulation could be changed in limitless ways by adjusting the ratios of the components to each other. An important feature of any formulation of the invention is that both the lipoic acid and inositol be present in a therapeutically effective amount. It is also important that the lipoic acid be released in a controlled manner which makes it possible to maintain therapeutic levels of lipoic acid over a substantially longer period of time as compared to a quick release formulation. A particularly preferred formulation will quickly obtain a therapeutic level of both active components and thereafter decrease the rate of release to closely match the rate at which the active components are being metabolized thereby maintaining a therapeutic level in the patient over a maximum period of time based on the amount of active components in the oral dosage formulation. Some general types of controlled release technology which might be used with the present invention are described below followed by specific preferred formulations. Although these technologies may be applied to both the lipoic acid and the inositol compound, it is more important to use such for the lipoic acid component.

### Controlled Release Technology

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

There are corporations with specific expertise in drug delivery technologies including controlled release oral formulations such as Alza Corporation and Elan Pharmaceuticals. A search of patents, published patent applications and related publications will provide those skilled in the art reading this disclosure with significant possible controlled release oral formulations. Examples include the formulations disclosed in any of the U.S. Pat. No. 5,637,320 issued Jun. 10, 1997; U.S. Pat. No. 5,505,962 issued Apr. 9, 1996; U.S. Pat. No. 5,641,745 issued Jun. 24, 1997; and U.S. Pat. No. 5,641,515 issued Jun. 24, 1997. Although specific formulations are disclosed here and in these patents the invention is more general than any specific formulation. This includes the discovery that by placing lipoic acid in a controlled release formulation which maintains therapeutic levels over substantially longer periods of time as compared to quick release formulations, improved unexpected results are obtained.

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods Theory and Applications, 1980 (CRC Press, Inc.).

Controlled release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

In rate-preprogrammed drug delivery systems, release of drug molecules from the delivery systems “preprogrammed” at specific rate profiles. This may be accomplished by system design, which controls the molecular diffusion of drug molecules in and/or across the barrier medium within or surrounding the delivery system. Fick's laws of diffusion are often followed.

In activation-modulated drug delivery systems, release of drug molecules from the delivery systems is activated by some physical, chemical or biochemical processes and/or facilitated by the energy supplied externally. The rate of drug release is then controlled by regulating the process applied, or energy input.

In feedback-regulated drug delivery systems, release of drug molecules from the delivery systems may be activated by a triggering event, such as a biochemical substance, in the body. The rate of drug release is then controlled by the concentration of triggering agent detected by a sensor in the feedback regulated mechanism.

In a site-targeting controlled-release drug delivery system, the drug delivery system targets the active molecule to a specific site or target tissue or cell. This may be accomplished, for example, by a conjugate including a site specific targeting moiety that leads the drug delivery system to the vicinity of a target tissue (or cell), a solubilizer that enables the drug delivery system to be transported to and preferentially taken up by a target tissue, and a drug moiety that is covalently bonded to the polymer backbone through a spacer and contains a cleavable group that can be cleaved only by a specific enzyme at the target tissue.

While a preferable mode of controlled release drug delivery will be oral, other modes of delivery of controlled release compositions according to this invention may be used. These include mucosal delivery, nasal delivery, ocular delivery, transdermal delivery, parenteral controlled release delivery, vaginal delivery, and intrauterine delivery.

There are a number of controlled release drug formulations that are developed preferably for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). Some of these formulations will now be discussed in more detail.

Enteric coatings are applied to tablets to prevent the release of drugs in the stomach either to reduce the risk of unpleasant side effects or to maintain the stability of the drug which might otherwise be subject to degradation of expose to the gastric environment. Most polymers that are used for this purpose are polyacids that function by virtue or the fact that their solubility in aqueous medium is pH-dependent, and they require conditions with a pH higher then normally encountered in the stomach.

One preferable type of oral controlled release structure is enteric coating of a solid or liquid dosage form. Enteric coatings promote the lipoates' remaining physically incorporated in the dosage form for a specified period when exposed to gastric juice. Yet the enteric coatings are designed to disintegrate in intestinal fluid for ready absorption. Delay of the lipoates' absorption is dependent on the rate of transfer through the gastrointestinal tract, and so the rate of gastric emptying is an important factor. Some investigators have reported that a multiple-unit type dosage form, such as granules, may be superior to a single-unit type. Therefore, in a preferable embodiment, the lipoates may be contained in an enterically coated multiple-unit dosage form. In a more preferable embodiment, the lipoate dosage form is prepared by spray-coating granules of a lipoate-enteric coating agent solid dispersion on an inert core material. These granules can result in prolonged absorption of the drug with good bioavailability.

Typical enteric coating agents include, but are not limited to, hydroxypropylmethylcellulose phthalate, methacryclic acid-methacrylic acid ester copolymer, polyvinyl acetate-phthalate and cellulose acetate phthalate. Akihiko Hasegawa, Application of solid dispersions of Nifedipine with enteric coating agent to prepare a sustained-release dosage form, Chem. Pharm. Bull. 33: 1615-1619 (1985). Various enteric coating materials may be selected on the basis of testing to achieve an enteric coated dosage form designed ab initio to have a preferable combination of dissolution time, coating thicknesses and diametral crushing strength. S. C. Porter et al., The Properties of Enteric Tablet Coatings Made From Polyvinyl Acetate-phthalate and Cellulose acetate Phthalate, J. Pharm. Pharmacol. 22:42p (1970).

On occasion, the performance of an enteric coating may hinge on its permeability. S. C. Porter et al., The Permeability of Enteric Coatings and the Dissolution Rates of Coated Tablets, J. Pharm. Pharmacol. 34: 5-8 (1981). With such oral drug delivery systems, the drug release process may be initiated by diffusion of aqueous fluids across the enteric coating. Investigations have suggested osmotic driven/rupturing affects as important release mechanisms from enteric coated dosage forms. Roland Bodmeier et al., Mechanical Properties of Dry and Wet Cellulosic and Acrylic Films Prepared from Aqueous Colloidal Polymer Dispersions used in the Coating of Solid Dosage Forms, Pharmaceutical Research, 11: 882-888 (1994).

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method. Akihiko Hasegawa, Super Saturation Mechanism of Drugs from Solid Dispersions with Enteric Coating Agents, Chem. Pharm. Bull. 36: 4941-4950 (1998). The solid dispersions may be also called solid-state dispersions. The term “coprecipitates” may also be used to refer to those preparations obtained by the solvent methods.

Solid dispersions may be used to improve the solubilities and/or dissolution rates of poorly water-soluble lipoates. Hiroshi Yuasa, et al., Application of the Solid Dispersion Method to the Controlled Release Medicine. III. Control of the Release Rate of Slightly Water-Soluble Medicine From Solid Dispersion Granules Chem. Pharm. Bull. 41:397-399 (1993). The solid dispersion method was originally used to enhance the dissolution rate of slightly water-soluble medicines by dispersing the medicines into water-soluble carriers such as polyethylene glycol or polyvinylpyraolidone, Hiroshi Yuasa, et al., Application of the Solid Dispersion Method to the Controlled Release of Medicine. IV. Precise Control of the Release Rate of a Water-Soluble Medicine by Using the Solid Dispersion Method Applying the Difference in the Molecular Weight of a Polymer, Chem. Pharm. Bull. 41:933-936 (1993).

The selection of the carrier may have an influence on the dissolution characteristics of the dispersed drug because the dissolution rate of a component from a surface may be affected by other components in a multiple component mixture. For example, a water-soluble carrier may result in a fast release of the drug from the matrix, or a poorly soluble or insoluble carrier may lead to a slower release of the drug from the matrix. The solubility of the lipoates may also be increased owing to some interaction with the carriers.

Examples of carriers useful in solid dispersions according to the invention include, but are not limited to, water-soluble polymers such as polyethylene glycol, polyvinylpyraolidone, or hydroxypropylmethyl-cellulose. Akihiko Hasegawa, Application of Solid Dispersions of Nifedipine with Enteric Coating Agent to Prepare a Sustained-release Dosage Form, Chem. Pharm. Bull. 33: 1615-1619 (1985).

Alternate carriers include phosphatidylcholine. Makiko Fujii, et al., The Properties of Solid Dispersions of Indomethacin, Ketoprofen and Flurbiprofen in Phosphatidylcholine, Chem. Pharm. Bull. 36:2186-2192 (1988). Phosphatidylcholine is an amphoteric but water-insoluble lipid, which may improve the solubility of otherwise insoluble lipoates in an amorphous state in phosphatidylcholine solid dispersions. See Makiko Fujii, et al., Dissolution of Bioavailibility of Phenytoin in Solid Dispersion with Phosphatidylcholine, Chem. Pharm. Bull 36:4908-4913 (1988).

Other carriers include polyoxyethylene hydrogenated castor oil. Katsuhiko Yano, et al., In-Vitro Stability and In-Vivo Absorption Studies of Colloidal Particles Formed From a Solid Dispersion System, Chem. Pharm. Bull 44:2309-2313 (1996). Poorly water-soluble lipoates may be included in a solid dispersion system with an enteric polymer such as hydroxypropylmethylcellulose phthalate and carboxymethylethylcellulose, and a non-enteric polymer, hydroxypropylmethylcellulose. See Toshiya Kai, et al., Oral Absorption Improvement of Poorly Soluble Drug Using Soluble Dispersion Technique, Chem. Pharm. Bull. 44:568-571 (1996). Another solid dispersion dosage form include incorporation of the drug of interest with ethyl cellulose and stearic acid in different ratios. Kousuke Nakano, et al., Oral Sustained-Release Cisplatin Preparations for Rats and Mice, J. Pharm. Pharmacol. 49:485-490 (1997).

There are various methods commonly known for preparing solid dispersions. These include, but are not limited to the melting method, the solvent method and the melting-solvent method.

In the melting method, the physical mixture of a drug in a water-soluble carrier is heated directly until it melts. The melted mixture is then cooled and solidified rapidly while rigorously stirred. The final solid mass is crushed, pulverized and sieved. Using this method a super saturation of a solute or drug in a system can often be obtained by quenching the melt rapidly from a high temperature. Under such conditions, the solute molecule may be arrested in solvent matrix by the instantaneous solidification process. A disadvantage is that many substances, either drugs or carriers, may decompose or evaporate during the fusion process at high temperatures. However, this evaporation problem may be avoided if the physical mixture is heated in a sealed container. Melting under a vacuum or blanket of an inert gas such as nitrogen may be employed to prevent oxidation of the drug or carrier.

The solvent method has been used in the preparation of solid solutions or mixed crystals of organic or inorganic compounds. Solvent method dispersions may prepared by dissolving a physical mixture of two solid components in a common solvent, followed by evaporation of the solvent. The main advantage of the solvent method is that thermal decomposition of drugs or carriers may be prevented because of the low temperature required for the evaporation of organic solvents. However, some disadvantages associated with this method are the higher cost of preparation, the difficulty in completely removing liquid solvent, the possible adverse effect of its supposedly negligible amount of the solvent on the chemical stability of the drug.

Another method of producing solid dispersions is the melting-solvent method. It is possible to prepare solid dispersions by first dissolving a drug in a suitable liquid solvent and then incorporating the solution directly into a melt of polyethylene glycol, obtainable below 70 degrees, without removing the liquid solvent. The selected solvent or dissolved lipoate may be selected such that the solution is not miscible with the melt of polyethylene glycol. The polymorphic form of the lipoate may then be precipitated in the melt. Such a unique method possesses the advantages of both the melting and solvent methods. Win Loung Chiou, et al., Pharmaceutical Applications of Solid Dispersion Systems, J. Pharm. Sci. 60:1281-1301 (1971).

Another controlled release dosage form is a complex between an ion exchange resin and the lipoates. Ion exchange resin-drug complexes have been used to formulate sustained-release products of acidic and basic drugs. In one preferable embodiment, a polymeric film coating is provided to the ion exchange resin-drug complex particles, making drug release from these particles diffusion controlled. See Y. Raghunathan et al., Sustained-released drug delivery system I: Coded ion-exchange resin systems for phenylpropanolamine and other drugs, J. Pharm. Sciences 70: 379-384 (1981).

Injectable micro spheres are another controlled release dosage form. Injectable micro spheres may be prepared by non-aqueous phase separation techniques, and spray-drying techniques. Micro spheres may be prepared using polylactic acid or copoly(lactic/glycolic acid). Shigeyuki Takada, Utilization of an Amorphous Form of a Water-Soluble GPIIb/IIIa Antagonist for Controlled Release From Biodegradable Micro spheres, Pharm. Res. 14:1146-1150 (1997), and ethyl cellulose, Yoshiyuki Koida, Studies on Dissolution Mechanism of Drugs from Ethyl Cellulose Microcapsules, Chem. Pharm. Bull. 35:1538-1545 (1987).

Other controlled release technologies that may be used in the practice of this invention are quite varied. They include SODAS, INDAS, IPDAS, MODAS, EFVAS, PRODAS, and DUREDAS. SODAS are multi particulate dosage forms utilizing controlled release beads. INDAS are a family of drug delivery technologies designed to increase the solubility of poorly soluble drugs. IPDAS are multi particulate tablet formation utilizing a combination of high density controlled release beads and an immediate release granulate. MODAS are controlled release single unit dosage forms. Each tablet consists of an inner core surrounded by a semipermeable multiparous membrane that controls the rate of drug release. EFVAS is an effervescent drug absorption system. PRODAS is a family of multi particulate formulations utilizing combinations of immediate release and controlled release mini-tablets. DUREDAS is a bilayer tablet formulation providing dual release rates within the one dosage form. Although these dosage forms are known to one of skill, certain of these dosage forms will now be discussed in more detail.

INDAS was developed specifically to improve the solubility and absorption characteristics of poorly water soluble drugs. Solubility and, in particular, dissolution within the fluids of the gastrointestinal tract is a key factor in determining the overall oral bioavailability of poorly water soluble drug. By enhancing solubility, one can increase the overall bioavailability of a drug with resulting reductions in dosage. INDAS takes the form of a high energy matrix tablet, production of which is comprised of two distinct steps: the adensosine analog in question is converted to an amorphous form through a combination of energy, excipients, and unique processing procedures.

Once converted to the desirable physical form, the resultant high energy complex may be stabilized by an absorption process that utilizes a novel polymer cross-linked technology to prevent recrystallization. The combination of the change in the physical state of the lipoate coupled with the solubilizing characteristics of the excipients employed enhances the solubility of the lipoate. The resulting absorbed amorphous drug complex granulate may be formulated with a gel-forming erodible tablet system to promote substantially smooth and continuous absorption.

IPDAS is a multi-particulate, tablet technology that may enhance the gastrointestinal tolerability of potential irritant and ulcerogenic drugs. Intestinal protection is facilitated by the multi-particulate nature of the IPDAS formulation which promotes dispersion of an irritant lipoate throughout the gastrointestinal tract. Controlled release characteristics of the individual beads may avoid high concentration of drug being both released locally and absorbed systemically. The combination of both approaches serves to minimize the potential harm of the lipoates with resultant benefits to patients.

IPDAS is composed of numerous high density controlled release beads. Each bead may be manufactured by a two step process that involves the initial production of a micromatrix with embedded lipoates and the subsequent coating of this micromatrix with polymer solutions that form a rate limiting semipermeable membrane in vivo. Once an IPDAS tablet is ingested, it may disintegrate and liberate the beads in the stomach. These beads may subsequently pass into the duodenum and along the gastrointestinal tract, preferably in a controlled and gradual manner, independent of the feeding state. Lipoate release occurs by diffusion process through the micromatrix and subsequently through the pores in the rate controlling semipermeable membrane. The release rate from the IPDAS tablet may be customized to deliver a drug-specific absorption profile associated with optimized clinical benefit. Should a fast onset of activity be necessary, immediate release granulate may be included in the tablet. The tablet may be broken prior to administration, without substantially compromising drug release, if a reduced dose is required for individual titration.

MODAS is a drug delivery system that may be used to control the absorption of water soluble lipoates. Physically MODAS is a non-disintegrating table formulation that manipulates drug release by a process of rate limiting diffusion by a semipermeable membrane formed in vivo. The diffusion process essentially dictates the rate of presentation of drug to the gastrointestinal fluids, such that the uptake into the body is controlled. Because of the minimal use of excipients, MODAS can readily accommodate small dosage size forms. Each MODAS tablet begins as a core containing active drug plus excipients. This core is coated with a solution of insoluble polymers and soluble excipients. Once the tablet is ingested, the fluid of the gastrointestinal tract may dissolve the soluble excipients in the outer coating leaving substantially the insoluble polymer. What results is a network of tiny, narrow channels connecting fluid from the gastrointestinal tract to the inner drug core of water soluble drug. This fluid passes through these channels, into the core, dissolving the drug, and the resultant solution of drug may diffuse out in a controlled manner. This may permit both controlled dissolution and absorption. An advantage of this system is that the drug releasing pores of the tablet are distributed over substantially the entire surface of the tablet. This facilitates uniform drug absorption reduces aggressive unidirectional drug delivery. MODAS represents a very flexible dosage form in that both the inner core and the outer semipermeable membrane may be altered to suit the individual delivery requirements of a drug. In particular, the addition of excipients to the inner core may help to produce a microenvironment within the tablet that facilitates more predictable release and absorption rates. The addition of an immediate release outer coating may allow for development of combination products.

Additionally, PRODAS may be used to deliver lipoates according to the invention. PRODAS is a multi particulate drug delivery technology based on the production of controlled release mini tablets in the size range of 1.5 to 4 mm in diameter. The PRODAS technology is a hybrid of multi particulate and hydrophilic matrix tablet approaches, and may incorporate, in one dosage form, the benefits of both these drug delivery systems.

In its most basic form, PRODAS involves the direct compression of an immediate release granulate to produce individual mini tablets that contain lipoates. These mini tablets are subsequently incorporated into hard gels and capsules that represent the final dosage form. A more beneficial use of this technology is in the production of controlled release formulations. In this case, the incorporation of various polymer combinations within the granulate may delay the release rate of drugs from each of the individual mini tablets. These mini tablets may subsequently be coated with controlled release polymer solutions to provide additional delayed release properties. The additional coating may be necessary in the case of highly water soluble drugs or drugs that are perhaps gastroirritants where release can be delayed until the formulation reaches more distal regions of the gastrointestinal tract. One value of PRODAS technology lies in the inherent flexibility to formulation whereby combinations of mini tablets, each with different release rates, are incorporated into one dosage form. As well as potentially permitting controlled absorption over a specific period, this also may permit targeted delivery of drug to specific sites of absorption throughout the gastrointestinal tract. Combination products also may be possible using mini tablets formulated with different active ingredients.

DUREDAS is a bilayer tableting technology that may be used in the practice of the invention. DUREDAS was developed to provide for two different release rates, or dual release of a drug from one dosage form. The term bilayer refers to two separate direct compression events that take place during the tableting process. In a preferable embodiment, an immediate release granulate is first compressed, being followed by the addition of a controlled release element which is then compressed onto this initial tablet. This may give rise to the characteristic bilayer seen in the final dosage form.

The controlled release properties may be provided by a combination of hydrophilic polymers. In certain cases, a rapid release of the lipoic acid may be desirable in order to facilitate a fast onset of therapeutic affect. Hence one layer of the tablet may be formulated as an immediate release granulate. By contrast, the second layer of the tablet may release the drug in a controlled manner, preferably through the use of hydrophilic polymers. This controlled release may result from a combination of diffusion and erosion through the hydrophilic polymer matrix.

A further extension of DUREDAS technology is the production of controlled release combination dosage forms. In this instance, two different lipoic acid compounds may be incorporated into the bilayer tablet and the release of drug from each layer controlled to maximize therapeutic affect of the combination.

The α-lipoic acid of the invention can be incorporated into any one of the aforementioned controlled released dosage forms, or other conventional dosage forms. The amount of α-lipoic acid contained in each dose can be adjusted, to meet the needs of the individual patient, and the indication. One of skill in the art and reading this disclosure will readily recognize how to adjust the level of α-lipoic acid and the release rates in a controlled release formulation, in order to optimize delivery of α-lipoic acid and its bioavailability.

Similarly, an inositol compound can be incorporated into any one of the aforementioned controlled released dosage forms, or other conventional dosage forms. The amount of inositol compound contained in each dose can be adjusted, to meet the needs of the individual patient, and the indication. One of skill in the art and reading this disclosure will readily recognize how to adjust the level of inositol compound and the release rates in a controlled release formulation, in order to optimize delivery of inositol compound and its bioavailability.

Oral Formulations

For oral delivery, a subject formulation will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

As one non-limiting example of a suitable oral formulation, lipoic acid and an inositol compound are formulated together with one or more pharmaceutical excipients and coated with an enteric coating, as described in U.S. Pat. No. 6,346,269. For example, a solution comprising a lipoic acid, an inositol compound, and a stabilizer is coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents for the active agent (lipoic acid and inositol compound) include aqueous solvents. Suitable stabilizers include alkali-metals and alkaline earth metals, bases of phosphates and organic acid salts and organic amines. The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™, copolymer of methyl acrylic acid/methyl methacrylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate(HPMCP), cellulose acetate phthalate (CAP), polyvinyl phthalic acetate (PVPA), Eudragit™ and shellac.

Suitable oral formulations also include lipoic acid and an inositol compound formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B. V.).

Suitable oral formulations also include lipoic acid and an inositol compound formulated with any of the following: a carrier such as Emisphere® (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (GlaxoSmithKline); SAVIT™ (Mistral Pharma Inc.); RingCap™ (Alza Corp.); Smartrix® (Smartrix Technologies, Inc.); SQZgel™ (MacroMed, Inc.); Geomatrix™ (Skye Pharma, Inc.); Oros® Tri-layer (Alza Corporation); and the like.

Also suitable for use are formulations such as those described in U.S. Pat. No. 6,296,842 (Alkermes Controlled Therapeutics, Inc.); U.S. Pat. No. 6,187,330 (Scios, Inc.); and the like.

Also suitable for use herein are formulations comprising an intestinal absorption enhancing agent. Suitable intestinal absorption enhancers include, but are not limited to, calcium chelators (e.g., citrate, ethylenediamine tetracetic acid); surfactants (e.g., sodium dodecyl sulfate, bile salts, palmitoylcarnitine, and sodium salts of fatty acids); toxins (e.g., zonula occludens toxin); and the like.

Treatment Methods

The present invention provides treatment methods, including methods for reducing serum glucose level in an individual; methods for treating diabetes; methods for treating insulin resistance; methods for treating disorders associated with or resulting from insulin resistance including polycystic ovary syndrome; and methods of treating metabolic syndrome. The methods generally involve administering to an individual in need thereof an effective amount of a subject inositol compound controlled release formulation, or a subject formulation comprising an inositol compound and a lipoic acid.

In many embodiments, multiple doses of a subject formulation are administered at various intervals and for various treatment durations. For example, a subject formulation is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). In some embodiments, a subject formulation is administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

In some embodiments, a subject formulation is administered following a meal, e.g., within 2 hours after a meal, e.g., from about 1 minute to about 2 hours after a meal. In other embodiments, a subject formulation is administered before a meal, e.g., from about 1 minute to about 30 minutes before a meal. In other embodiments, an active agent is administered as needed to lower blood glucose levels, e.g., a subject formulation is administered within about 1 minute to about 30 minutes following a blood glucose measurement that indicates that the blood glucose level exceeds the normal range.

In other embodiments, a subject formulation is administered following appearance of a symptom of a disorder (e.g., diabetes, metabolic disorder, etc.), e.g., a subject formulation is administered 5 minutes, 10 minutes, 15 minutes, 30 minutes, 1 hour, 2 hours, 4 hours, 8 hours, 16 hours, 24 hours, or 48 hours, after appearance of a symptom such as higher than normal serum glucose levels, elevated blood pressure, and the like.

Methods of Reducing Blood Glucose Levels

In some embodiments, the present invention provides methods of reducing blood glucose levels in an individual. The methods are useful for treating diabetes, e.g., Type 1 diabetes, Type 2 diabetes, gestational diabetes. Accordingly, the present invention further provides methods of treating diabetes, including methods of treating Type I diabetes in an individual, and methods of treating Type II diabetes. The methods generally involve administering to an individual in need thereof an effective amount of a subject inositol compound controlled release formulation, or a subject formulation comprising an inositol compound and a lipoic acid.

In some embodiments, an effective amount of a subject formulation is an amount that is effective to reduce a blood glucose level in an individual by at least about 10%, at least about 15%, at least about 20%, at least about 25%, at least about 30%, at least about 40%, or at least about 50% when compared to the blood glucose levels in the absence of the formulation. In some embodiments, an effective amount of a subject formulation is an amount that is effective to reduce blood glucose levels to within a normal range. Normal blood glucose levels are typically in the range of from about 70 mg/dL to about 110 mg/dL before a meal (e.g., a fasting blood glucose level); and less than 120 mg/dL 2 hours after a meal.

Methods of Treating Insulin Resistance

The present invention further provides methods of treating insulin resistance in an individual, e.g., methods of increasing the response of an individual, or cells in an individual, to insulin. The methods generally involve administering to an individual in need thereof an effective amount of a subject inositol compound controlled release formulation, or a subject formulation comprising an inositol compound and a lipoic acid. A subject formulation increases the sensitivity of an individual to insulin.

An effective amount of a subject formulation increases the response of an individual to insulin, e.g., endogenously produced insulin or administered insulin. In some embodiments, a subject formulation is administered in conjunction with insulin (e.g., co-administered with insulin, or administered before or after insulin administration).

An effective amount of a subject formulation reduces blood glucose levels in response to insulin, e.g., reduces blood glucose levels by at least about 10%, at least about 15%, at least about 20%, at least about 25%, at least about 30%, at least about 40%, or at least about 50% when compared to the blood glucose levels in the absence of the formulation and in response to insulin. In some embodiments, an effective amount of a subject formulation is an amount that is effective to reduce blood glucose levels to within a normal range in response to insulin.

Insulin resistance can lead to a variety of disorders, including, e.g., polycystic ovarian syndrome, cardiovascular disease, essential hypertension, and non-alcoholic fatty liver disease. The present invention provides methods of treating such disorders, and reducing the risk that an individual will develop such disorders.

Methods of Treating Metabolic Syndrome

The present invention further provides methods of treating metabolic syndrome in an individual. The methods generally involve administering to an individual in need thereof an effective amount of a subject inositol compound controlled release formulation, or a subject formulation comprising an inositol compound and a lipoic acid. Individuals who have metabolic syndrome include individuals meeting 3 or more of the following criteria: 1) Abdominal obesity: Men: Waist circumference >40 inches; Women: Waist circumference >35 inches; 2) Fasting glucose ≧110-126 mg/dL; 3) Blood pressure ≧130/80 mm Hg; 4) Triglycerides ≧150 mg/dL; and 5) high density lipoproteins (HDL) cholesterol: Men <40 mg/dL; Women <50 mg/dL.

An effective amount of a subject formulation is in many embodiments an amount that reduces an indicator of metabolic syndrome by at least about 10%, at least about 15%, at least about 20%, at least about 25%, at least about 30%, at least about 40%, or at least about 50%, or more, compared to an individual having metabolic syndrome who has not been treated with a subject formulation. Thus, e.g., an effective amount of a subject formulation reduces one or more of abdominal obesity (waist circumference), fasting glucose levels, blood pressure, plasma or serum triglyceride levels, and HDL cholesterol levels by at least about 10%, at least about 15%, at least about 20%, at least about 25%, at least about 30%, at least about 40%, or at least about 50%, or more, compared to an individual having metabolic syndrome who has not been treated with a subject formulation.

Subjects Suitable for Treatment

Subjects suitable for treatment with a subject formulation include individuals who have been diagnosed with diabetes mellitus. Such individuals include those having a fasting blood glucose level greater than about 126 mg/dL. Such individuals include those having blood glucose levels of greater than about 200 mg/dL following a two-hour glucose tolerance test (75 g anhydrous glucose orally). Also suitable for treatment with a formulation or method of the present invention are individuals polycystic ovary syndrome. Also suitable for treatment with a formulation or method of the present invention are individuals with metabolic syndrome. Also suitable for treatment with a formulation or a subject method are individuals with a fasting blood insulin level of greater than about 15 μU/ml.

Lipoic Acid and an Inositol Compound

In some embodiments, the invention provides a combination therapy method using combined effective amounts of a lipoic acid and an inositol compound, the method comprising co-administering to an individual (e.g., in a single formulation, i.e., where the lipoic acid and the inositol compound are co-formulated) in need thereof a) a dosage of a lipoic acid containing an amount of from about 50 mg to about 1000 mg, administered orally tid, bid, qd, qod, or as needed; and b) a dosage of an inositol compound containing an amount of from about 50 mg to about 1000 mg, administered orally tid, bid, qd, qod, or as needed, for the desired treatment duration. In many embodiments, the lipoic acid and the inositol compound are co-administered in the same formulation.

In some embodiments, the invention provides a combination therapy method using combined effective amounts of a lipoic acid and an inositol compound, the method comprising co-administering to an individual in need thereof a) a dosage of a lipoic acid containing an amount of 150 mg, administered orally tid, bid, qd, qod, or as needed; and b) a dosage of an inositol compound containing an amount of 150 mg, administered orally tid, bid, qd, qod, or as needed, for the desired treatment duration. In many embodiments, the lipoic acid and the inositol compound are co-administered in the same formulation.

In some embodiments, the invention provides a combination therapy method using combined effective amounts of a lipoic acid and an inositol compound, the method comprising co-administering to an individual in need thereof a) a dosage of a lipoic acid containing an amount of 300 mg, administered orally tid, bid, qd, qod, or as needed; and b) a dosage of an inositol compound containing an amount of 300 mg, administered orally tid, bid, qd, qod, or as needed, for the desired treatment duration. In many embodiments, the lipoic acid and the inositol compound are co-administered in the same formulation.

In some embodiments, the invention provides a combination therapy method using combined effective amounts of a lipoic acid and an inositol compound, the method comprising co-administering to an individual in need thereof a) a dosage of a lipoic acid containing an amount of 150 mg, administered orally tid, bid, qd, qod, or as needed; and b) a dosage of an inositol compound containing an amount of 150 mg D-chiro-inositol, administered orally tid, bid, qd, qod, or as needed, for the desired treatment duration. In many embodiments, the lipoic acid and the D-chiro-inositol are co-administered in the same formulation.

In some embodiments, the invention provides a combination therapy method using combined effective amounts of a lipoic acid and an inositol compound, the method comprising co-administering to an individual in need thereof a) a dosage of a lipoic acid containing an amount of 300 mg, administered orally tid, bid, qd, qod, or as needed; and b) a dosage of an inositol compound containing an amount of 300 mg D-chiro-inositol, administered orally tid, bid, qd, qod, or as needed, for the desired treatment duration. In many embodiments, the lipoic acid and the D-chiro-inositol are co-administered in the same formulation.

In some embodiments, the invention provides a combination therapy method using combined effective amounts of a lipoic acid and an inositol compound, the method comprising co-administering to an individual in need thereof a) a dosage of a lipoic acid containing an amount of 150 mg, administered orally tid, bid, qd, qod, or as needed; and b) a dosage of an inositol compound containing an amount of 150 mg pinitol, administered orally tid, bid, qd, qod, or as needed, for the desired treatment duration. In many embodiments, the lipoic acid and the pinitol are co-administered in the same formulation.

Lipoic Acid, an Inositol Compound, and an Additional Anti-Diabetic Agent

In some embodiments, the invention provides a combination therapy method using combined effective amounts of a lipoic acid and an inositol compound, the method comprising co-administering to an individual in need thereof a) a dosage of a lipoic acid containing an amount of from about 50 mg to about 1000 mg, administered orally tid, bid, qd, qod, or as needed; b) a dosage of an inositol compound containing an amount of from about 50 mg to about 1000 mg, administered orally tid, bid, qd, qod, or as needed; and c) a dosage of an additional anti-diabetic agent selected from a thiazolidinedione, a sulfonylurea, a biguanide, a PPAR agonist, a DPP-IV inhibitor, and an α-glucosidase inhibitor, for the desired treatment duration. In many embodiments, the lipoic acid and the inositol compound are co-administered in the same formulation. In some embodiments, the lipoic acid, the inositol compound, and the additional anti-diabetic agent are co-administered in the same formulation.

In some embodiments, the invention provides a combination therapy method using combined effective amounts of a lipoic acid and an inositol compound, the method comprising co-administering to an individual in need thereof a) a dosage of a lipoic acid containing an amount of from about 50 mg to about 1000 mg, administered orally tid, bid, qd, qod, or as needed; b) a dosage of an inositol compound containing an amount of from about 50 mg to about 1000 mg, administered orally tid, bid, qd, qod, or as needed; and c) a dosage of an α-glucosidase inhibitor containing an amount of from about 10 mg to about 100 mg miglitol, administered orally tid, e.g., 10 mg, 15 mg, 20 mg, 25 mg, 30 mg, 40 mg, 50 mg, 60 mg, 70 mg, 75 mg, 80 mg, 90 mg, or 100 mg miglitol, administered orally tid, for the desired treatment duration.

In some embodiments, the invention provides a combination therapy method using combined effective amounts of a lipoic acid and an inositol compound, the method comprising co-administering to an individual in need thereof a) a dosage of a lipoic acid containing an amount of from about 50 mg to about 1000 mg, administered orally tid, bid, qd, qod, or as needed; b) a dosage of an inositol compound containing an amount of from about 50 mg to about 1000 mg, administered orally tid, bid, qd, qod, or as needed; and c) a dosage of an α-glucosidase inhibitor containing an amount of from about 10 mg to about 100 mg acarbose, administered orally tid, e.g., 10 mg, 15 mg, 20 mg, 25 mg, 30 mg, 40 mg, 50 mg, 60 mg, 70 mg, 75 mg, 80 mg, 90 mg, or 100 mg acarbose, administered orally tid, for the desired treatment duration.

In some embodiments, the invention provides a combination therapy method using combined effective amounts of a lipoic acid and an inositol compound, the method comprising co-administering to an individual in need thereof a) a dosage of a lipoic acid containing an amount of from about 50 mg to about 1000 mg, administered orally tid, bid, qd, qod, or as needed; b) a dosage of an inositol compound containing an amount of from about 50 mg to about 1000 mg, administered orally tid, bid, qd, qod, or as needed; and c) a dosage of a biguanide containing an amount of from about 250 mg to about 1000 mg metformin HCl, e.g., 500 mg, administered orally bid or tid, for the desired treatment duration.

In some embodiments, the invention provides a combination therapy method using combined effective amounts of a lipoic acid and an inositol compound, the method comprising co-administering to an individual in need thereof a) a dosage of a lipoic acid containing an amount of from about 50 mg to about 1000 mg, administered orally tid, bid, qd, qod, or as needed; b) a dosage of an inositol compound containing an amount of from about 50 mg to about 1000 mg, administered orally tid, bid, qd, qod, or as needed; and c) a dosage of a thiazolidinedione containing an amount of 2 mg, 4 mg, or 8 mg rosiglitazone, administered orally qd or bid, for the desired treatment duration.

Further Combinations

In some embodiments, any of the above-described treatment regimens is further modified to include administering a lipid-soluble thiamine. In some embodiments, the lipid-soluble thiamine is benfotiamine.

### Therapeutic Indications/Lipoic Acid

Formulations of the present invention can be used to obtain a wide range of desirable effects. Particularly the formulations of the invention are useful in treating essentially any disease state or symptom which is treatable by long term administration of antioxidants. Further, formulations of the invention can be used in treating patients with abnormally low levels of any inositol. Still further, the invention can be used in the treatment of diseases which involve carbohydrate metabolism and blood glucose disposal which includes various forms of diabetes. In addition, the inventions can be used in the treatment of diabetic polyneuropathy. Further, the invention is useful in the treatment of various adverse effects on the eyes and skin when the adverse effect are due to high levels of free radicals which can be dissipated by the presence of antioxidants or high levels of serum glucose which can be reduced by stimulating basal glucose transport. Maintaining substantially constant levels of lipoic acid provides a long term antioxidant effect which assists in immunomodulation and can result in improved liver and kidney function. Because of the long term antioxidant effect in the circulatory system the present invention has a variety of beneficial effects on the cardiovascular system. Administering the inositol compound is useful in the alleviation of neurodegenerative diseases related to diabetes. A patient infected with HIV can benefit from the enhanced effect obtained on the immune system.

Because of the very minimal toxicity of both lipoic acid and inositol the formulation can be given to a wide range of patients which have different conditions from mild to serious without fear of adverse effects. Further, the controlled release formulations taught here are even safer than quick release formulations in that serum levels obtained are low compared to quick release formulations. One mild side effect experienced by some patients taking controlled release lipoic acid is mild headaches over the first few days. The headaches have not been observed with quick release formulations of lipoic acid. Patients treated with vasodilators experience the same mild headaches over the first days of treatment. The headaches are believed to be caused by the vasodilator effect allowing increased blood flow to the brain. Accordingly, controlled release formulations of the invention can be used as a vasodilator to treat patients with angina. Controlled release lipoic acid can be administered only with the inositol compound or along with a conventional vasodilator, e.g. with a nitroglycerin pill or transdermal patch.

The data provided here do not show specific treatments of many of the diseases or symptoms mentioned above. However, the invention is believed to be responsible for obtaining a wide range of beneficial effects particularly when the controlled release formulation is administered to patient's (e.g. on consecutive days) over long periods of time, i.e. weeks, months and years. By maintaining substantially constant therapeutic levels of lipoic acid and inositol in the blood over very long periods of time a range of desirable physiological results are obtained. Stated differently by continually maintaining the constant therapeutic serum levels of the powerful antioxidant and keeping a patient's blood glucose level within a more desirable range the adverse effects obtained from free radicals and high fluctuating glucose levels are avoided.

### Therapeutic Indications/Inositol

There is no known toxicity in humans from inositol taken orally. People have taken hundreds of milligrams daily without any harmful effect, although some may become more stimulated than others.

Subjects suitable for treatment with an agent or method of the present invention include individuals who have been diagnosed with diabetes mellitus. Such individuals include those having a fasting blood glucose level greater than about 126 mg/dL. Such individuals include those having blood glucose levels of greater than about 200 mg/dL following a two-hour glucose tolerance test (75 g anhydrous glucose orally). Also suitable for treatment with an agent or method of the present invention are individuals with metabolic syndrome (which consists of obesity, hypertension, dyslipidemia) and insulin resistance. Also suitable for treatment with a subject agent or a subject method are individuals with a fasting blood insulin level of greater than about 15 μU/ml.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

In a first step, racemic α-lipoic acid is screened to a particle size range of 150 to 450 microns. The racemic α-lipoic acid is added to a granulator. Examples of granulators include a Bohle (Bristol, Pa.) granulator and a Glatt (Ramsey, N.J.) fluid bed granulator. The racemic α-lipoic acid particles become the cores for a coated particle. The cores are coated with a 30% w/w aqueous dispersion of EUDRAGIT™ (NE30 D, methacrylic acid ester) and talc. This yields coated particles with a dried coating weight equal to about 10% of the total weight of the coated particle. The inlet air temperature is kept at a temperature of 25° C. After drying, the coated particles are screened using a 40 mesh screen.

The resulting, free-flowing particles are then blended and directly compressed using a tableting press according to the following formula:
Racemic α-lipoic acid, coated particles 81%
METHOCEL™ K 110 10%
(methylcellulose)
Microcrystalline cellulose 5%
Stearic Acid 3%
Micronized silica 0.5%
Magnesium Stearate 0.5%

The resulting tablet is a sustained release formulation.

### Example 2

In a first step, R-(+)-α-lipoic acid is screened to a particle size range of 150 to 450 microns. The R-(+)-α-lipoic acid is then added to a fluid bed granulator. The R-(+)-α-lipoic acid particles become the cores for a coated particle. The cores are coated with a 30% w/w aqueous dispersion of EUDRAGIT™ (NE30 D, methacrylic acid ester) and talc. This yields coated particles with a dried coating weight equal to about 10% of the total weight of the coated particle. The inlet air temperature is kept at a temperature of 25° C. After drying, the coated particles are screened using a 40 mesh screen.

The resulting, free-flowing particles are then blended and directly compressed using a tableting press according to the following formula:
R-(+)-α-lipoic acid, coated particles81%
METHOCEL™ K 110 10%
(methylcellulose)
Microcrystalline cellulose 5%
Stearic Acid 3%
Micronized silica 0.5%
Magnesium Stearate 0.5%

The resulting tablet is a sustained release formulation.

### Example 3

In a first step, R-(+)-α-lipoic acid is screened to a particle size range of 150 to 450 microns. The R-(+)-α-lipoic acid is then added to a fluid bed granulator. The R-(+) -α-lipoic acid particles become the cores for a coated particle. EUDRAGIT™ (L/S 100, methacrylic acid ester) is dissolved in isopropyl alcohol to form a 15% w/w solution. Triethyl citrate, talc, and water are additionally added to the solution. Total solids content of the resulting mixture is 9.6% w/w. This yields coated particles with a dried coating weight equal to about 10% of the total weight of the coated particle. The inlet air temperature is kept at a temperature of 25° C. After drying, the coated particles are screened using a 40 mesh screen.

The resulting, free-flowing particles are then blended and directly compressed using a tableting press according to the following formula:
R-(+)-α-lipoic acid, coated particles 81%
METHOCEL™ K100 5%
(methylcellulose)
Microcrystalline cellulose 5%
Stearic Acid 3%
Micronized silica 0.5%
Magnesium Stearate 0.5%

The resulting tablet is protected from the harsh acid environment of the stomach, and is delivered to the small intestine where it is gradually released.

### Example 4

In a first step, racemic α-lipoic acid is screened to a particle size range of 150 to 450 microns. The racemic α-lipoic acid is then added to a fluid bed granulator. The racemic α-lipoic acid particles become the cores for a coated particle. EUDRAGIT™ (L/S 100, methacrylic acid ester) is dissolved in isopropyl alcohol to form a 15% w/w solution. Triethyl citrate, talc, and water are additionally added to the solution. Total solids content of the resulting mixture is 9.6% w/w. This yields coated particles with a dried coating weight equal to about 10% of the total weight of the coated particle. The inlet air temperature is kept at a temperature of 25 deg C. After drying, the coated particles are screened using a 40 mesh screen.

The resulting, free-flowing particles are then blended and directly compressed using a tableting press according to the following formula:
Racemic α-lipoic acid, coated particles 81%
METHOCEL™ K100 5%
(methylcellulose)
Microcrystalline cellulose 5%
Stearic Acid 3%
Micronized silica 0.5%
Magnesium Stearate 0.5%

The resulting tablet is protected from the harsh acid environment of the stomach, and is delivered to the small intestine where it is gradually released.

### Example 5

A preblend of 98% w/w CARBOPOL7 934 (B.F. Goodrich Chemical, lightly cross-linked acrylic acid allyl sucrose copolymer) and 2% w/w micronized silica is prepared. To this mixture, racemic α-lipoic acid, METHOCEL™ K100, stearic acid, and lactose are added according to the following formula:
Racemic α-lipoic acid preblend 70%
CARBOPOL™ 934/silica preblend 10%
METHOCEL™ K 100 10%
stearic acid 5%
lactose 5%

The resulting mixture is tableted using a direct compression tableting press to form a bioadhesive formulation.

### Example 6

A preblend of 98% w/w R-(+)-α-lipoic acid and 2% w/w CAB-O-SIL™ micronized silica is formed. To this mixture is added guar gum (AQUALON™ G-3), polyvinylpyraolidone (PVP), calcium carbonate, stearic acid, lactose, and magnesium stearate in the following amounts:
R-(+)-α-lipoic acid/CAB-O-SIL7 blend49.5%
guar gum (AQUALON7 G-3)30%
polyvinylpyrrolidone (PVP) 5%
calcium carbonate 5%
stearic acid 5%
lactose 5%
magnesium stearate 0.5%

The resulting mixture is tableted using a direct compression tableting press to form a sustained release caplet formulation.

### Example 7

| Item | | | Theoretical | Unit of |
|---|---|---|---|---|
| No. | Item Description | Percent | Quantity | Measure |
| 1. | α-Lipoic Acid | 60 | 4800.0 | g |
| 2. | Microcrystalline Cellulose, | 18 | 1440.0 | g |
| | NF (Avicel PH 101) | | | |
| 3. | Aquacoat CPD (30% w/w) | 15* | 4000.0* | g |
| 4. | Povidone K29/32, USP | 3 | 240.0 | g |
| 5. | Carbopol 974P | 2.5 | 200.0 | g |
| 6. | Talc, USP | 1 | 80.0 | g |
| 7. | Magnesium Stearate, NF | 0.5 | 40.0 | g |
| 8. | Purified Water, USP | | — | g |
| N/A | TOTAL | 100 | 8000.0 | g |

*Quantity indicates amount of dispersion to be used in granulating. Actual Solids Content-1200 g - 15% is based on solids content

Before formulating a check should be made of the room and equipment in order to verify that the cleaning procedure has been performed and approved. Weigh and charge α-Lipoic Acid (Item 1) and Avicel PH 101, (Item 2) in a Hobart Mixer and mix for two (2) minutes with the mixer speed set at 1 or 2. Granulate the Step 2 material by slowly adding Aquacoat CPD (Item 3) until granules are formed. Add additional Purified Water, USP (Item 8) if required, and mix until the granules are formed. Mixer Speed Setting remains at 1-2. Spread the granulation evenly from Step 3 on paper-lined trays and load them into the oven. Dry at 40ECV5EC for two (2) hours. Check LOD and record moisture content. If LOD is more than 2%, continue drying until LOD is below 2%. Pass the dried material from Step 5 through a size 14 mesh screen, hand held or using a Quadro Comil. Charge the Step 6 granulation into a V-blender. Charge the Step 7 blend in blender with Povidone K29/32, USP (Item 4) and Carbopol 974P (Item 5) and mix for five (5) minutes. Charge the V-blender with Talc (Item 6) and Magnesium Stearate, NF (Item 7) and blend for three (3) minutes. Empty the blend from the V-blender into a properly labeled tared PE-lined container and record the weights in Step 11. Theoretical weight of blend: 8000.0 g. Lower Limit 95% and Upper Limit 102%. Any discrepancy from these established limits must be reported to Production and Quality Assurance. Any discrepancy must be appropriately investigated and documented. Hold the blend in the in-process Q.C. Hold area for further processing. Using the amounts shown above will result in sufficient formulations to produce above 16,000 300 mg tablets.

### Example 8

A controlled release oral dosage form of racemic α-lipoic acid was administered to a group of volunteers. Each dose consisted of a tablet containing 300 mg of racemic α-lipoic acid, compounded with calcium phosphate, starch, cellulose ethers, polycarboxylic acid, and magnesium stearate. The 300 mg tablets used with these patients were tablets prepared in a manner as described above in Example 7. Each patient was given two 300 mg tablets in the morning before eating and one 300 mg tablet within 6 to 8 hours.

The results were as follows:

| | | | Average | |
|---|---|---|---|---|
| | | | Glucose | |
| | | | Levels | |
| Patient No. | Sex (M/F) | Age | Before | After |
| 1 | M | 47 | 240 | 150 |
| 2 | F | 46 | 225 | 120 |
| 3 | M | 45 | 155 | 130 |
| 4 | M | 67 | 155 | 95 |
| 5 | F | 47 | 175 | 195 |
| 6 | M | 82 | 138 | 129 |
| 7 | M | 48 | 174 | 119 |
| 8 | M | 71 | 150 | 90 |

As can be seen from Table 1, the average glucose level before treatment with the controlled release lipoic acid was 176.5 mg/dl. After treatment with the controlled release lipoic acid, the average glucose level was 128.5 mg/dl, a average decrease of 48 mg/dl.

### Example 9

A controlled release oral dosage form of racemic α-lipoic acid was administered to a group of volunteers. Each dose consisted of a tablet containing 300 mg of racemic α-lipoic acid, compounded with calcium phosphate, starch, cellulose ethers, polycarboxylic acid, and magnesium stearate. The 300 mg tablets used with these patients were tablets prepared in a manner as described above in Example 7 and dosed in the same manner described in Example 7.

The results were as follows:

| | | | Average | |
|---|---|---|---|---|
| | | | Glucose | |
| | | | Levels | |
| Patient No. | Sex (M/F) | Age | Before | After |
| 1 | M | 62 | 400 | 140 |
| 2 | F | 65 | 300 | 149 |
| 3 | F | 51 | 325 | 185 |

As can be seen from Table 2, the average glucose level before treatment with the controlled release lipoic acid was 342 mg/dl. After treatment with the controlled release lipoic acid, the average glucose level was 158 mg/dl, a average decrease of 184 mg/dl.

### Example 10

Fourteen human volunteers described below were administered controlled release lipoic acid formulations of the present invention. The formulations were prepared in a manner such as that described in Example 7 above. Each patient was dosed with two 300 mg tablets in the morning before eating and one 300 mg tablet approximately six hours thereafter. In some instances some patients were dosed with additional medications as indicated. These results demonstrate the improved results with the lipoic acid controlled release formulations of the invention alone or in combination with other pharmaceutically active compositions.

CR ALA Tablet Study

| | | | | Average Glucose Levels | | | Percent | |
|---|---|---|---|---|---|---|---|---|
| Patient # | Type | Description | Age | Before | After | Change | Change | Comments |
| 1 | Type 2 | Glucophage 850 mg 3× | 51 | 220 | 110 | −110 | −50% | |
| 2 | type 2 | Insulin/Glucophage | 70 | 168 | 112 | −56 | −33% | |
| 3 | Type 2 | Insulin/Oral Meds | 54 | 175 | 120 | −55 | −31% | Cut meds in half and 9 to 7 A1C |
| 4 | Type 2 | Glucophage 500 mg | 65 | 135 | 114 | −21 | −16% | |
| | | 2× Day | | | | | | |
| 6 | type 2 | Diet & Exercise | 46 | 189 | 131 | −58 | −31% | Dr. did not have to put on drugs and drop |
| | | | | | | | | A1C from 8.3 to 6.2 |
| 7 | Type 2 | GlucophageXL | 67 | 135 | 90 | −45 | −33% | |
| 8 | Type 2 | Insulin/Glucophage | 46 | 300 | 200 | −100 | −33% | |
| 10 | Type 2 | Insulin/Oral Meds | 72 | 185 | 135 | −50 | −27% | |
| 11 | Type 2 | Insulin | 72 | 135 | 87 | −48 | −36% | |
| 12 | Type 2 | Glucophage/Glucotrol | 79 | 225 | 140 | −85 | −38% | |
| 13 | Type 2 | Diet & Exercise | 59 | 145 | 111 | −35 | −24% | |
| 14 | Type 2 | “Insulin, 15 unix 2×” | 51 | 325 | 191 | −134 | −41% | |
| | | AVERAGE = | 186 | 128 | −57 | −29% | | |
| 5 | Normal | Severe polyneuropathy | #N/A | #N/A | #N/A | #N/A | #N/A | Eliminated all neuropath |

### Example 11

In a first step, both a racemic α-lipoic acid and a pinitol are screened to a particle size range of 150 to 450 microns. The racemic α-lipoic acid and the pinitol are then added to a fluid bed granulator. The particles of racemic α-lipoic acid and pinitol become the cores for a coated particle. EUDRAGIT7 (L/S 100, methacrylic acid ester) is dissolved in isopropyl alcohol to form a 15% w/w solution. Triethyl citrate, talc, and water are additionally added to the solution. Total solids content of the resulting mixture is 9.6% w/w. This yields coated particles with a dried coating weight equal to about 10% of the total weight of the coated particle. The inlet air temperature is kept at a temperature of 25° C. After drying, the coated particles are screened using a 40 mesh screen.

The resulting, free-flowing particles are then blended and directly compressed using a tableting press according to the following formula:

Racemic α-lipoic acid, coated particles 41%
Pinitol 40%
METHOCEL7 K100 5%
(methylcellulose)
Microcrystalline cellulose 5%
Stearic Acid 3%
Micronized silica 0.5%
Magnesium Stearate 0.5%

### Example 12

In a first step, both a racemic α-lipoic acid and pinitol are screened to a particle size range of 150 to 450 microns. The racemic α-lipoic acid and pinitol are then added to a fluid bed granulator. The particles of racemic α-lipoic acid and pinitol become the cores for a coated particle. EUDRAGIT™ (L/S 100, methacrylic acid ester) is dissolved in isopropyl alcohol to form a 15% w/w solution. Triethyl citrate, talc, and water are additionally added to the solution. Total solids content of the resulting mixture is 9.6% w/w. This yields coated particles with a dried coating weight equal to about 10% of the total weight of the coated particle. The inlet air temperature is kept at a temperature of 25° C. After drying, the coated particles are screened using a 40 mesh screen.

The resulting, free-flowing particles are then blended and directly compressed using a tableting press according to the following formula:
Racemic a-lipoic acid, coated particles 36%
Pinitol 45%
METHOCEL™ K100 5%
(methylcellulose)
Microcrystalline cellulose 5%
Stearic Acid 3%
Micronized silica 0.5%
Magnesium Stearate 0.5%

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An oral dosage formulation, comprising:
a therapeutically effective amount of lipoic acid;
a therapeutically effective amount of an inositol compound; and
an excipient material.

2. The formulation of claim 1, wherein the lipoic acid comprises a racemic mixture of enantiomers.

3. The formulation of claim 1, wherein the lipoic acid comprises 80% or more R-(+) enantiomer of lipoic acid with 20% or less being the S-(−) enantiomer.

4. The formulation of claim 1, wherein the lipoic acid comprises a substantially pure R-(+) enantiomer of lipoic acid.

5. The formulation of claim 1, wherein the inositol compound is selected from D-chiro-inositol, a D-chiro-inositol-phosphate, pinitol, ciceritol, 1D-2-O-alpha-D-galactopyranose, and a fagopyritol.

6. The formulation of claim 1, wherein the formulation is characterized by releasing a first portion of the lipoic acid and the inositol compound sufficient to obtain a therapeutic level at a first rate substantially equivalent to a release rate of a quick release formulation and releasing a remaining portion of the lipoic acid and the inositol compound at a controlled rate which is below a release rate of a quick release formulation.

7. The formulation of claim 6, wherein the first portion of the inositol compound and the lipoic acid is from about 10% to about 50% of the inositol compound and lipoic acid in the formulation.

8. The formulation of claim 7, wherein the first portion of the inositol compound and lipoic acid is from about 20% to about 30% of the inositol compound and lipoic acid in the formulation.

9. The formulation of claim 8, wherein the first portion of the inositol compound and the lipoic acid is about 25% of the inositol compound and the lipoic acid in the formulation.

10. The formulation of claim 5, wherein the controlled rate maintains the therapeutic level of both the inositol compound and the lipoic acid for a period which is 10% or more longer as compared to a quick release formulation.

11. The formulation of claim 5, wherein the controlled rate maintains the therapeutic level of both the inositol compound and the lipoic acid for a period which is 50% or more longer as compared to a quick release formulation.

12. The formulation of claim 5, wherein the controlled rate maintains the therapeutic level of both the inositol compound and the lipoic acid for a period which is 100% or more longer as compared to a quick release formulation.

13. The formulation of claim 5, wherein the controlled rate maintains the therapeutic level of both the inositol compound and the lipoic acid for a period which is 200% or more longer as compared to a quick release formulation.

14. The formulation of claim 1, further comprising an orally active antidiabetic chosen from a sulfonylurea, a biguanide, PPARγ agonist, a PPARα/γ dual agonist, a thiazolidinedione, a dipeptidyl peptidase IV inhibitor, and an α-glucosidase inhibitor.

15. The formulation of claim 1, further comprising a lipid-soluble thiamine.

16. The formulation of claim 15, wherein the lipid-soluble thiamine is benfotiamine.

17. The formulation of claim 1, further comprising metformin hydrochloride.

18. The formulation of claim 1, wherein the lipoic acid is present as a racemic mixture of R-(+) and S-(−) enantiomers and the therapeutic level is maintained over a period of four hours or more.

19. The formulation of claim 1, wherein the lipoic acid is present as substantially pure R-(+) enantiomer and the therapeutic level is maintained over a period of four hours or more and further wherein the inositol is chosen from D-chiro inositol and pinitol.

20. The formulation of claim 5, wherein the controlled rate is a rate of about 25% or less per hour slower than a quick release formulation.

21. The formulation of claim 5, wherein the controlled rate is a rate of about 50% or less per hour slower than a quick release formulation.

22. A method of treatment, comprising:
orally administering to a patient a formulation comprising an inositol compound and lipoic acid; and
repeating the administering on three or more consecutive days thereby maintain a therapeutic level of both the inositol compound and lipoic acid in the patient's circulatory system over a therapeutically effective period of time on three or more consecutive days.

23. The method of claim 22, wherein the therapeutic level is maintained over a period of time which is 10% or more than that obtained with a quick release formulation and further wherein the repeating is over thirty or more consecutive days.

24. The method of claim 22, wherein the therapeutic level is maintained over a period of time which is 100% or more than that obtained with a quick release formulation and further wherein the repeating is over thirty or more consecutive days.

25. The method of claim 22, wherein the therapeutic level of lipoic acid is a level sufficient to obtain measurable vasodilation in a human patient.

26. The method of claim 22, wherein the therapeutic level is a level sufficient to obtain a measurable reduction in a human patient's serum glucose level.

27. A method of reducing a human patient's serum glucose level, comprising:
administering a therapeutically effective amount of an orally active antidiabetic selected from the group consisting of a sulfonylurea, a biguanide, PPARγ agonist, a PPARα/γ dual agonist, a thiazolidinedione, a dipeptidyl peptidase IV inhibitor, and an α-glucosidase inhibitor; and
administering an oral formulation of an inositol compound and lipoic acid.

28. The method of claim 27, further comprising:
repeatedly administering the antidiabetic and the formulation of the inositol compound and the lipoic acid on a daily basis for five or more days.

29. The method of claim 27, wherein the antidiabetic is metformin hydrochloride which is administered in an amount in a range of about 500 mg to about 1,000 mg per day.

30. A method of treating a human patient, comprising:
administering to a human patient a biphasic formulation of an inositol compound and lipoic acid which formulation is characterized by maintaining a therapeutic level of the inositol compound and lipoic acid in the patient's circulatory system over a period of time greater than that obtained with a quick release formulation; and
repeating the administering on three or more consecutive days thereby maintain a therapeutic level of both the inositol compound and the lipoic acid in the patient's circulatory system over a therapeutically effective period of time on three or more consecutive days.

31. A method of treating diabetes mellitus, insulin resistance, the metabolic syndrome, polycystic ovary syndrome comprising the steps of:
orally administering to a diabetic human patient a therapeutically effective amount of a formulation comprising lipoic acid and an inositol compound; and
repeating the administering on three or more consecutive days thereby maintain a therapeutic level of both the inositol compound and the lipoic acid in the patient's circulatory system over a therapeutically effective period of time on three or more consecutive days.

32. The method of claim 31, wherein the lipoic acid is a racemic mixture of enantiomers.

33. The method of claim 31, wherein the lipoic acid is a substantially pure R-(+) enantiomer of lipoic acid.

34. The method of claim 31, further comprising:
orally administering metformin hydrochloride in an amount in a range of from about 500 mg to about 1,000 mg per day; and
repeating the administration on three or more consecutive days.

35. A method of treating insulin resistance in an individual, the method comprising orally administering to a diabetic human patient a therapeutically effective amount of a formulation comprising lipoic acid and an inositol compound.

36. The method of claim 35, further comprising repeating the administering on three or more consecutive days thereby maintain a therapeutic level of both the inositol compound and the lipoic acid in the patient's circulatory system over a therapeutically effective period of time on three or more consecutive days.
